SEQUENCE LISTING (19 sequences)

SEQ ID NO: 1 - protein, 632 aa, Artificial sequence
  Binding domain
NYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLD

SEQ ID NO: 2 - protein, 13 aa, Artificial sequence
  SpyTag
AHIVMVDAYKPTK

SEQ ID NO: 3 - protein, 38 aa, Artificial sequence
  p53dim domain
GGEYFTLQIRGRERFEEFREKNEALELKDAQAGKEPGG

SEQ ID NO: 4 - protein, 115 aa, Artificial sequence
  SpyCatcher
AMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 5 - protein, 31 aa, Artificial sequence
  Twin-Strep-tag
SAWSHPQFEKGGGSGGGSGGSSAWSHPQFEK

SEQ ID NO: 6 - protein, 5 aa, Artificial sequence
  Flexible Linker
GGGGS

SEQ ID NO: 7 - protein, 14 aa, Artificial sequence
  Helix-forming Peptide Linker
LVGEAAAKEAAAKA

SEQ ID NO: 8 - protein, 808 aa, Artificial sequence
  Catenated VAR2CSA recombinant protein AXVB
MAHIVMVDAYKPTKGGGGSGGEYFTLQIRGRERFEEFREKNEALELKDAQAGKEPGGVDNYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDGTAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 9 - protein, 1442 aa, Artificial sequence
  Catenated VAR2CSA recombinant protein VAXVB
MNYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDGSAHIVMVDAYKPTKGGGGSGGEYFTLQIRGRERFEEFREKNEALELKDAQAGKEPGGVDNYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDGTAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 10 - protein, 1437 aa, Artificial sequence
  Catenated VAR2CSA recombinant protein AVXVB
MAHIVMVDAYKPTKNYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDGSGGEYFTLQIRGRERFEEFREKNEALELKDAQAGKEPGGVDNYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDGTAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 11 - DNA, 2427 bp, Artificial sequence
  Nucleic acid molecule axvb
atggcgcacattgttatggtggacgcgtacaaaccgaccaagggtggcggtggcagcggtggcgagtatttcaccctgcagatccgtggccgtgaacgtttcgaggaatttcgtgagaaaaacgaagcgctggagctgaaagatgcgcaagcgggcaaggagccgggtggcgtcgacaactacatcaaaggcgatccgtatttcgcggaatacgcgaccaagctgagctttattctgaacagcagcgacgcgaacaacccgagcgagaaaatccagaagaacaacgatgaagtgtgcaactgcaacgagagcggtattgcgagcgttgagcaggaacaaatcagcgacccgagcagcaacaaaacctgcattacccacagcagcatcaaggcgaacaagaaaaaggtttgcaaacacgtgaagctgggcgttcgtgagaacgacaaggatctgcgtgtttgcgtgattgagcacaccagcctgagcggtgtggaaaactgctgctgccaggactttctgcgtatcctgcaagaaaactgcagcgataacaaaagcggtagcagcagcaacggcagctgcaacaacaagaaccaggaagcgtgcgagaaaaacctggagaaggttctggcgagcctgaccaactgctacaaatgcgacaaatgcaagagcgaacaaagcaaaaagaacaacaagaactggatttggaaaaagagcagcggcaaagaaggtggcctgcagaaggagtatgcgaacaccatcggtctgccgccgcgtacccaaagcctgtgcctggtggtttgcctggatgaaaaaggtaaaaagacccaggagctgaagaacatccgtaccaacagcgaactgctgaaagagtggatcattgcggcgttccacgagggcaaaaacctgaagccgagccacgagaagaagaacgacgataacggtaaaaagctgtgcaaggcgctggagtatagctttgcggactacggtgatctgattaaaggcaccagcatctgggacaacgaatacaccaaggatctggagctgaacctgcagaaaattttcggcaagctgttccgtaagtacatcaaaaagaacaacaccgcggaacaagacaccagctatagcagcctggatgaactgcgtgagtcctggtggaacaccaacaaaaagtacatctggctggcgatgaaacacggtgcgggcatgaacagcaccacctgctgcggtgatggcagcgtgaccggtagcggcagcagctgcgacgatatcccgaccattgatctgatcccgcagtatctgcgtttcctgcaagaatgggttgagcacttttgcaaacagcgtcaagaaaaagttaagccggtgattgagaactgcaaaagctgcaaggaaagcggtggcacctgcaacggtgaatgcaagaccgagtgcaaaaacaagtgcgaggtgtacaaaaagttcatcgaagactgcaaaggtggcgatggcaccgcgggcagcagctgggttaagcgttgggaccagatttacaagcgttatagcaaatacatcgaggatgcgaagcgtaaccgtaaagcgggcaccaagaactgcggtccgagcagcaccaccaacgcggcggaaaacaaatgcgtgcaaagcgacattgatagcttctttaagcacctgatcgacattggcctgaccaccccgagcagctacctgagcattgttctggacgataacatttgcggtgcggacaaggcgccgtggaccacctataccacctacaccaccaccgaaaaatgcaacaaggaaaccgataaaagcaagctgcagcaatgcaacaccgcggtggttgtgaacgttccgagcccgctgggtaacaccccgcacggctacaaatatgcgtgccagtgcaagatcccgaccaacgaggaaacctgcgacgatcgtaaagagtatatgaaccaatggagctgcggtagcgcgcgtaccatgaaacgtggctataagaacgacaactacgaactgtgcaaatataacggcgttgatgtgaagccgaccaccgtgcgtagcaacagcagcaagctggacggtaccgcgatggttgataccctgagcggtctgagcagcgagcagggtcaaagcggcgacatgaccattgaggaagatagcgcgacccacatcaaattcagcaagcgtgacgaagatggtaaagagctggcgggcgcgaccatggaactgcgtgacagcagcggcaagaccattagcacctggatcagcgacggccaggtgaaagatttctacctgtatccgggcaagtacacctttgttgaaaccgcggcgccggatggttatgaagtggcgaccgcgattacctttaccgttaacgagcagggtcaagttaccgtgaacggtaaagcgaccaagggcgatgcgcacatctaa

SEQ ID NO: 12 - DNA, 4329 bp, Artificial sequence
  Nucleic acid molecule vaxvb
atgaactacatcaaaggcgatccgtatttcgcggaatacgcgaccaagctgagctttattctgaacagcagcgacgcgaacaacccgagcgagaaaatccagaagaacaacgatgaagtgtgcaactgcaacgagagcggtattgcgagcgttgagcaggaacaaatcagcgacccgagcagcaacaaaacctgcattacccacagcagcatcaaggcgaacaagaaaaaggtttgcaaacacgtgaagctgggcgttcgtgagaacgacaaggatctgcgtgtttgcgtgattgagcacaccagcctgagcggtgtggaaaactgctgctgccaggactttctgcgtatcctgcaagaaaactgcagcgataacaaaagcggtagcagcagcaacggcagctgcaacaacaagaaccaggaagcgtgcgagaaaaacctggagaaggttctggcgagcctgaccaactgctacaaatgcgacaaatgcaagagcgaacaaagcaaaaagaacaacaagaactggatttggaaaaagagcagcggcaaagaaggtggcctgcagaaggagtatgcgaacaccatcggtctgccgccgcgtacccaaagcctgtgcctggtggtttgcctggatgaaaaaggtaaaaagacccaggagctgaagaacatccgtaccaacagcgaactgctgaaagagtggatcattgcggcgttccacgagggcaaaaacctgaagccgagccacgagaagaagaacgacgataacggtaaaaagctgtgcaaggcgctggagtatagctttgcggactacggtgatctgattaaaggcaccagcatctgggacaacgaatacaccaaggatctggagctgaacctgcagaaaattttcggcaagctgttccgtaagtacatcaaaaagaacaacaccgcggaacaagacaccagctatagcagcctggatgaactgcgtgagtcctggtggaacaccaacaaaaagtacatctggctggcgatgaaacacggtgcgggcatgaacagcaccacctgctgcggtgatggcagcgtgaccggtagcggcagcagctgcgacgatatcccgaccattgatctgatcccgcagtatctgcgtttcctgcaagaatgggttgagcacttttgcaaacagcgtcaagaaaaagttaagccggtgattgagaactgcaaaagctgcaaggaaagcggtggcacctgcaacggtgaatgcaagaccgagtgcaaaaacaagtgcgaggtgtacaaaaagttcatcgaagactgcaaaggtggcgatggcaccgcgggcagcagctgggttaagcgttgggaccagatttacaagcgttatagcaaatacatcgaggatgcgaagcgtaaccgtaaagcgggcaccaagaactgcggtccgagcagcaccaccaacgcggcggaaaacaaatgcgtgcaaagcgacattgatagcttctttaagcacctgatcgacattggcctgaccaccccgagcagctacctgagcattgttctggacgataacatttgcggtgcggacaaggcgccgtggaccacctataccacctacaccaccaccgaaaaatgcaacaaggaaaccgataaaagcaagctgcagcaatgcaacaccgcggtggttgtgaacgttccgagcccgctgggtaacaccccgcacggctacaaatatgcgtgccagtgcaagatcccgaccaacgaggaaacctgcgacgatcgtaaagagtatatgaaccaatggagctgcggtagcgcgcgtaccatgaaacgtggctataagaacgacaactacgaactgtgcaaatataacggcgttgatgtgaagccgaccaccgtgcgtagcaacagcagcaagctggacggatccgcgcacattgttatggtggacgcgtacaaaccgaccaagggtggcggtggcagcggtggcgagtatttcaccctgcagatccgtggccgtgaacgtttcgaggaatttcgtgagaaaaacgaagcgctggagctgaaagatgcgcaagcgggcaaggagccgggtggcgtcgacaactacatcaaaggcgatccgtatttcgcggaatacgcgaccaagctgagctttattctgaacagcagcgacgcgaacaacccgagcgagaaaatccagaagaacaacgatgaagtgtgcaactgcaacgagagcggtattgcgagcgttgagcaggaacaaatcagcgacccgagcagcaacaaaacctgcattacccacagcagcatcaaggcgaacaagaaaaaggtttgcaaacacgtgaagctgggcgttcgtgagaacgacaaggatctgcgtgtttgcgtgattgagcacaccagcctgagcggtgtggaaaactgctgctgccaggactttctgcgtatcctgcaagaaaactgcagcgataacaaaagcggtagcagcagcaacggcagctgcaacaacaagaaccaggaagcgtgcgagaaaaacctggagaaggttctggcgagcctgaccaactgctacaaatgcgacaaatgcaagagcgaacaaagcaaaaagaacaacaagaactggatttggaaaaagagcagcggcaaagaaggtggcctgcagaaggagtatgcgaacaccatcggtctgccgccgcgtacccaaagcctgtgcctggtggtttgcctggatgaaaaaggtaaaaagacccaggagctgaagaacatccgtaccaacagcgaactgctgaaagagtggatcattgcggcgttccacgagggcaaaaacctgaagccgagccacgagaagaagaacgacgataacggtaaaaagctgtgcaaggcgctggagtatagctttgcggactacggtgatctgattaaaggcaccagcatctgggacaacgaatacaccaaggatctggagctgaacctgcagaaaattttcggcaagctgttccgtaagtacatcaaaaagaacaacaccgcggaacaagacaccagctatagcagcctggatgaactgcgtgagtcctggtggaacaccaacaaaaagtacatctggctggcgatgaaacacggtgcgggcatgaacagcaccacctgctgcggtgatggcagcgtgaccggtagcggcagcagctgcgacgatatcccgaccattgatctgatcccgcagtatctgcgtttcctgcaagaatgggttgagcacttttgcaaacagcgtcaagaaaaagttaagccggtgattgagaactgcaaaagctgcaaggaaagcggtggcacctgcaacggtgaatgcaagaccgagtgcaaaaacaagtgcgaggtgtacaaaaagttcatcgaagactgcaaaggtggcgatggcaccgcgggcagcagctgggttaagcgttgggaccagatttacaagcgttatagcaaatacatcgaggatgcgaagcgtaaccgtaaagcgggcaccaagaactgcggtccgagcagcaccaccaacgcggcggaaaacaaatgcgtgcaaagcgacattgatagcttctttaagcacctgatcgacattggcctgaccaccccgagcagctacctgagcattgttctggacgataacatttgcggtgcggacaaggcgccgtggaccacctataccacctacaccaccaccgaaaaatgcaacaaggaaaccgataaaagcaagctgcagcaatgcaacaccgcggtggttgtgaacgttccgagcccgctgggtaacaccccgcacggctacaaatatgcgtgccagtgcaagatcccgaccaacgaggaaacctgcgacgatcgtaaagagtatatgaaccaatggagctgcggtagcgcgcgtaccatgaaacgtggctataagaacgacaactacgaactgtgcaaatataacggcgttgatgtgaagccgaccaccgtgcgtagcaacagcagcaagctggacggtaccgcgatggttgataccctgagcggtctgagcagcgagcagggtcaaagcggcgacatgaccattgaggaagatagcgcgacccacatcaaattcagcaagcgtgacgaagatggtaaagagctggcgggcgcgaccatggaactgcgtgacagcagcggcaagaccattagcacctggatcagcgacggccaggtgaaagatttctacctgtatccgggcaagtacacctttgttgaaaccgcggcgccggatggttatgaagtggcgaccgcgattacctttaccgttaacgagcagggtcaagttaccgtgaacggtaaagcgaccaagggcgatgcgcacatctaa

SEQ ID NO: 13 - DNA, 4314 bp, Artificial sequence
  Nucleic acid molecule avxvb
atggcgcacattgttatggtggacgcgtacaaaccgaccaagaactacatcaaaggcgatccgtatttcgcggaatacgcgaccaagctgagctttattctgaacagcagcgacgcgaacaacccgagcgagaaaatccagaagaacaacgatgaagtgtgcaactgcaacgagagcggtattgcgagcgttgagcaggaacaaatcagcgacccgagcagcaacaaaacctgcattacccacagcagcatcaaggcgaacaagaaaaaggtttgcaaacacgtgaagctgggcgttcgtgagaacgacaaggatctgcgtgtttgcgtgattgagcacaccagcctgagcggtgtggaaaactgctgctgccaggactttctgcgtatcctgcaagaaaactgcagcgataacaaaagcggtagcagcagcaacggcagctgcaacaacaagaaccaggaagcgtgcgagaaaaacctggagaaggttctggcgagcctgaccaactgctacaaatgcgacaaatgcaagagcgaacaaagcaaaaagaacaacaagaactggatttggaaaaagagcagcggcaaagaaggtggcctgcagaaggagtatgcgaacaccatcggtctgccgccgcgtacccaaagcctgtgcctggtggtttgcctggatgaaaaaggtaaaaagacccaggagctgaagaacatccgtaccaacagcgaactgctgaaagagtggatcattgcggcgttccacgagggcaaaaacctgaagccgagccacgagaagaagaacgacgataacggtaaaaagctgtgcaaggcgctggagtatagctttgcggactacggtgatctgattaaaggcaccagcatctgggacaacgaatacaccaaggatctggagctgaacctgcagaaaattttcggcaagctgttccgtaagtacatcaaaaagaacaacaccgcggaacaagacaccagctatagcagcctggatgaactgcgtgagtcctggtggaacaccaacaaaaagtacatctggctggcgatgaaacacggtgcgggcatgaacagcaccacctgctgcggtgatggcagcgtgaccggtagcggcagcagctgcgacgatatcccgaccattgatctgatcccgcagtatctgcgtttcctgcaagaatgggttgagcacttttgcaaacagcgtcaagaaaaagttaagccggtgattgagaactgcaaaagctgcaaggaaagcggtggcacctgcaacggtgaatgcaagaccgagtgcaaaaacaagtgcgaggtgtacaaaaagttcatcgaagactgcaaaggtggcgatggcaccgcgggcagcagctgggttaagcgttgggaccagatttacaagcgttatagcaaatacatcgaggatgcgaagcgtaaccgtaaagcgggcaccaagaactgcggtccgagcagcaccaccaacgcggcggaaaacaaatgcgtgcaaagcgacattgatagcttctttaagcacctgatcgacattggcctgaccaccccgagcagctacctgagcattgttctggacgataacatttgcggtgcggacaaggcgccgtggaccacctataccacctacaccaccaccgaaaaatgcaacaaggaaaccgataaaagcaagctgcagcaatgcaacaccgcggtggttgtgaacgttccgagcccgctgggtaacaccccgcacggctacaaatatgcgtgccagtgcaagatcccgaccaacgaggaaacctgcgacgatcgtaaagagtatatgaaccaatggagctgcggtagcgcgcgtaccatgaaacgtggctataagaacgacaactacgaactgtgcaaatataacggcgttgatgtgaagccgaccaccgtgcgtagcaacagcagcaagctggacggatccggtggcgagtatttcaccctgcagatccgtggccgtgaacgtttcgaggaatttcgtgagaaaaacgaagcgctggagctgaaagatgcgcaagcgggcaaggagccgggtggcgtcgacaactacatcaaaggcgatccgtatttcgcggaatacgcgaccaagctgagctttattctgaacagcagcgacgcgaacaacccgagcgagaaaatccagaagaacaacgatgaagtgtgcaactgcaacgagagcggtattgcgagcgttgagcaggaacaaatcagcgacccgagcagcaacaaaacctgcattacccacagcagcatcaaggcgaacaagaaaaaggtttgcaaacacgtgaagctgggcgttcgtgagaacgacaaggatctgcgtgtttgcgtgattgagcacaccagcctgagcggtgtggaaaactgctgctgccaggactttctgcgtatcctgcaagaaaactgcagcgataacaaaagcggtagcagcagcaacggcagctgcaacaacaagaaccaggaagcgtgcgagaaaaacctggagaaggttctggcgagcctgaccaactgctacaaatgcgacaaatgcaagagcgaacaaagcaaaaagaacaacaagaactggatttggaaaaagagcagcggcaaagaaggtggcctgcagaaggagtatgcgaacaccatcggtctgccgccgcgtacccaaagcctgtgcctggtggtttgcctggatgaaaaaggtaaaaagacccaggagctgaagaacatccgtaccaacagcgaactgctgaaagagtggatcattgcggcgttccacgagggcaaaaacctgaagccgagccacgagaagaagaacgacgataacggtaaaaagctgtgcaaggcgctggagtatagctttgcggactacggtgatctgattaaaggcaccagcatctgggacaacgaatacaccaaggatctggagctgaacctgcagaaaattttcggcaagctgttccgtaagtacatcaaaaagaacaacaccgcggaacaagacaccagctatagcagcctggatgaactgcgtgagtcctggtggaacaccaacaaaaagtacatctggctggcgatgaaacacggtgcgggcatgaacagcaccacctgctgcggtgatggcagcgtgaccggtagcggcagcagctgcgacgatatcccgaccattgatctgatcccgcagtatctgcgtttcctgcaagaatgggttgagcacttttgcaaacagcgtcaagaaaaagttaagccggtgattgagaactgcaaaagctgcaaggaaagcggtggcacctgcaacggtgaatgcaagaccgagtgcaaaaacaagtgcgaggtgtacaaaaagttcatcgaagactgcaaaggtggcgatggcaccgcgggcagcagctgggttaagcgttgggaccagatttacaagcgttatagcaaatacatcgaggatgcgaagcgtaaccgtaaagcgggcaccaagaactgcggtccgagcagcaccaccaacgcggcggaaaacaaatgcgtgcaaagcgacattgatagcttctttaagcacctgatcgacattggcctgaccaccccgagcagctacctgagcattgttctggacgataacatttgcggtgcggacaaggcgccgtggaccacctataccacctacaccaccaccgaaaaatgcaacaaggaaaccgataaaagcaagctgcagcaatgcaacaccgcggtggttgtgaacgttccgagcccgctgggtaacaccccgcacggctacaaatatgcgtgccagtgcaagatcccgaccaacgaggaaacctgcgacgatcgtaaagagtatatgaaccaatggagctgcggtagcgcgcgtaccatgaaacgtggctataagaacgacaactacgaactgtgcaaatataacggcgttgatgtgaagccgaccaccgtgcgtagcaacagcagcaagctggacggtaccgcgatggttgataccctgagcggtctgagcagcgagcagggtcaaagcggcgacatgaccattgaggaagatagcgcgacccacatcaaattcagcaagcgtgacgaagatggtaaagagctggcgggcgcgaccatggaactgcgtgacagcagcggcaagaccattagcacctggatcagcgacggccaggtgaaagatttctacctgtatccgggcaagtacacctttgttgaaaccgcggcgccggatggttatgaagtggcgaccgcgattacctttaccgttaacgagcagggtcaagttaccgtgaacggtaaagcgaccaagggcgatgcgcacatctaa

SEQ ID NO: 14 - DNA, 363 bp, Artificial sequence
  Heavy chain variable region of single-chain fragment variable antibody
gaggtgaagctggtggaaagcggcggcggactggtgaaacctggaggcagcctgaagctcagctgcgccgctagcggatttacatttagcaactacgccatgagctgggtgcggcagagccccgagaggcgcctggaatgggtcgctgagatcagcatcaccggcagatacacctactaccccgacaccgttacaggccggttcaccatcagccgggacaacgccaagaacaccctgtacctggagatgagttctctgagatctgaagataccgccatgtactactgcaccagagagggctacgactacgccccttcttggttcgcctactggggacagggaaccctggtcaccgtgtccgcc

SEQ ID NO: 15 - DNA, 336 bp, Artificial sequence
  Light chain variable region of single-chain fragment variable antibody
gacgtggtgatgacccagacacctctgagcctgcctgtgtctctgggcgaccaggccagcatcagctgtagaagcggccagaccctggtgcacagaaatggcatcacatacctggaatggtatctgcagaagcctggccaatctccaaagctgctgatctacaaggtgtccaaccggttcagcggcgtgcccgatagattcagcggcagcggctccggcaccgacttcaccctgaaaatctccagagtggaagccgaggatctgggaatctactattgcttccagggctctcacgtgcctagaacattcggcggaggtacaaagctggagattaag

SEQ ID NO: 16 - protein, 536 aa, Artificial sequence
  Chimeric antigen receptor
MALPVTALLLPLALLLHAARPDVVMTQTPLSLPVSLGDQASISCRSGQTLVHRNGITYLEWYLQKPGQSPKLLIYKVSNRFSGVPDRFSGSGSGTDFTLKISRVEAEDLGIYYCFQGSHVPRTFGGGTKLEIKGGGGSGGGGSGGGGSEVKLVESGGGLVKPGGSLKLSCAASGFTFSNYAMSWVRQSPERRLEWVAEISITGRYTYYPDTVTGRFTISRDNAKNTLYLEMSSLRSEDTAMYYCTREGYDYAPSWFAYWGQGTLVTVSATTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 632 aa, Artificial sequence; identical to SEQ ID NO: 1
  rVAR2 protein

SEQ ID NO: 18 - protein, 250 aa, Artificial sequence
  Anti-CD19 ScFv
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 19 - protein, 15 aa, Artificial sequence
  Connection sequence
GGGGSGGGGSGGGGS

(19) United States
(12) Patent Application Publication
(10) Pub. No.: US 2025/0073269 A1
(43) Pub. Date: Mar. 6, 2025

(54) CATENATED VAR2CSA RECOMBINANT PROTEIN, PREPARATION METHOD THEREFOR, AND USE THEREOF

(71) Applicant: CAS LAMVAC (GUANGZHOU) BIOMEDICAL TECHNOLOGY CO., LTD, Guangdong, Guangdong (CN)

(72) Inventors: Wen Hu, Guangdong (CN); Zhu Tao, Guangdong (CN); Wenzhong Guo, Guangdong (CN); Wenting Ding, Guangdong (CN); Li Qin, Guangdong (CN); Xiaoping Chen, Guangdong (CN)

(21) Appl. No.: 18/727,302

(22) PCT Filed: Jun. 9, 2022
(86) PCT No.: PCT/CN2022/097809

§ 371 Date: Jul. 8, 2024

(30) Foreign Application Priority Data
Jan. 7, 2022 (CN) 202210012869.2

(51) Int. Cl. A61K 35/17; A61K 39/00; A61P 35/00; C07K 14/47; C07K 14/705; C07K 14/725; C07K 16/18; C12N 5/0783; C12N 15/86

(52) U.S. Cl. CPC A61K 35/17; A61K 40/11; A61K 40/31; A61P 35/00; C07K 14/4746; C07K 14/7051; C07K 14/70517; C07K 14/70521; C07K 16/18; C12N 5/0636; C12N 15/86; A61K 2239/13; A61K 2239/17; A61K 2239/21; C07K 2317/622; C07K 2319/02; C07K 2319/03; C07K 2319/33; C12N 2740/15043

(57) ABSTRACT

Provided is a catenated VAR2CSA recombinant protein, a preparation therefor and a use thereof. The catenated VAR2CSA recombinant protein comprises a binding domain, SpyTag, a p53dim structural domain, and SpyCatcher, which are randomly arranged. The binding domain comprises a structural domain, in the VAR2CSA protein, binding to placenta-like chondroitin sulfate A. The catenated VAR2CSA recombinant protein has high stability and affinity to the tumor-specific antigen placenta-like chondroitin sulfate A, and can be effectively used in the field of tumor immunotherapy, such as immune cell therapy.

### TECHNICAL FIELD

The present application belongs to the field of cell immunotherapy and relates to a catenated VAR2CSA recombinant protein, a preparation method therefor, and use thereof.

### BACKGROUND

In recent years, immune cell therapies represented by chimeric antigen receptor T (CAR-T) cell technology have achieved great success in the clinical treatment of hematological tumors, and the ones for the treatment of solid tumors are also being explored (June et al., Science. 2018, 359(6382): 1361-1365). As clinical studies continue to be conducted, more and more researchers have found that there are still 10-20% of patients with B-cell lymphoma and B-lymphoblastic leukemia who do not respond to Anti-CD19 CAR-T therapies (Maude et al., N Engl J Med. 2018, 378(5): 439-448). In some of the clinical trials, the relapse rate in the patients treated with Anti-CD19 CAR-T therapies was up to over 50% after one year of treatment (Park et al., N Engl J Med. 2018, 378(5): 449-459). The main reasons for these phenomena include the objective presence of CD19-negative tumors in patients, poor in vivo persistence of the CAR-T cells, the down-regulation or loss of the membrane expression of CD19 target antigens of cancer cells due to mutations of the CD19 target antigens, etc.

To address the challenges of poor CAR-T cell persistence and the lack or loss of CD19 antigens in B-lymphoblastic tumors, researchers have taken the following measures: 1) blocking chimeric antigen receptor (CAR) ubiquitination to enhance endosomal CAR signaling and promote CAR-T cell persistence (Li et al., Immunity. 2020, 53(2):456-470.e6); 2) generating a CAR with a low affinity for CD19 antigens to improve the proliferation capacity of CAR-T cells and enhance the persistence of CAR-T cells (Ghorashian et al., Nat Med. 2019, 25(9): 1408-1414.); 3) constructing a dual-targeting CAR-T cell, e.g., targeting both CD19 and CD20 antigens (Zah et al., Cancer Immunol Res. 2016, 4(6): 498-508) to reduce the chances of antigen escape by B-cell tumors. Furthermore, the toxic and side effects of CAR-T cell therapy, for example, cytokine release syndrome (CRS) or immune effector cell-associated neurotoxicity syndrome (ICANS), and other safety issues that threaten patients' survival should not be ignored. In addition to strengthening medical grading and management (Neelapu S S. Hematol Oncol. 2019, 37 Suppl 1: 48-52), the innovative design of a CAR-T cell system at its source (Jaspers and Brentjens. Pharmacol Ther. 2017, 178: 83-91) may be the only way to solve the problem once and for all.

In conclusion, the design and manufacture of a broad-spectrum (multi-targeted) chimeric antigen receptor cell system with high security, great stability and durable anti-tumor activity is the key to achieving a greater breakthrough in the field of immune cell therapy.

### SUMMARY

The present application provides a catenated VAR2CSA recombinant protein, a preparation therefor, and use thereof. As compared to a normal VAR2CSA recombinant protein (wild-type), the catenated VAR2CSA recombinant protein has higher protein stability and stronger affinity for the tumor-specific antigen placenta-like chondroitin sulfate A (pl-CSA) and can be used in the preparation of an anti-tumor drug such as a chimeric antigen receptor cell.

In a first aspect, the present application provides a catenated VAR2CSA recombinant protein. The catenated VAR2CSA recombinant protein includes a binding domain, SpyTag, a p53dim domain and SpyCatcher that are randomly arranged and linked. The binding domain includes a domain, in the VAR2CSA protein, binding to pl-CSA.

In the present application, the binding domain in the VAR2CSA protein capable of recognizing and binding to the tumor-specific antigen pl-CSA is randomly combined and linked with SpyTag, a p53dim domain and SpyCatcher to form a fusion protein. Due to the formation of an isopeptide bond by spontaneous amide-bond condensation of a short peptide SpyTag with its protein ligand SpyCatcher, a covalent coupling reaction occurs within a fusion protein molecule (N-terminus and C-terminus) or between fusion protein molecules, thereby resulting in protein molecule cyclization and catenation and ultimately forming a “collar” multimeric recombinant protein. Through the comparison with a monomeric VAR2CSA recombinant protein (rVAR2), catenation significantly improves the protein stability and protein affinity for the tumor-specific antigen pl-CSA. The anti-tumor effect of a CAR-T cell system using the catenated VAR2CSA recombinant protein as the navigation system is comprehensively enhanced, and with a low cytokine secretion level, it is speculated that such a CAR-T cell system has a low risk of toxic side effects such as cytokine release syndrome clinically. Therefore, the catenated VAR2CSA recombinant protein can be effectively applied to the field of tumor immunotherapy such as immune cell therapy.

Preferably, a polypeptide sequence of the binding domain includes a sequence shown in SEQ ID NO. 1.

Preferably, a polypeptide sequence of SpyTag includes a sequence shown in SEQ ID NO. 2.

Preferably, a polypeptide sequence of the p53dim domain includes a sequence shown in SEQ ID NO. 3.

Preferably, a polypeptide sequence of SpyCatcher includes a sequence shown in SEQ ID NO. 4.

SEQ ID NO. 1:
NYIKGDPYFAEYATKLSFILNSSDANNPSEKIQKNNDEVCNCNESGIAS
VEQEQISDPSSNKTCITHSSIKANKKKVCKHVKLGVRENDKDLRVCVIE
HTSLSGVENCCCQDFLRILQENCSDNKSGSSSNGSCNNKNQEACEKNLE
KVLASLTNCYKCDKCKSEQSKKNNKNWIWKKSSGKEGGLQKEYANTIGL
PPRTQSLCLVVCLDEKGKKTQELKNIRTNSELLKEWIIAAFHEGKNLKP
SHEKKNDDNGKKLCKALEYSFADYGDLIKGTSIWDNEYTKDLELNLQKI
FGKLFRKYIKKNNTAEQDTSYSSLDELRESWWNTNKKYIWLAMKHGAGM
NSTTCCGDGSVTGSGSSCDDIPTIDLIPQYLRFLQEWVEHFCKQRQEKV
KPVIENCKSCKESGGTCNGECKTECKNKCEVYKKFIEDCKGGDGTAGSS
WVKRWDQIYKRYSKYIEDAKRNRKAGTKNCGPSSTTNAAENKCVQSDID
SFFKHLIDIGLTTPSSYLSIVLDDNICGADKAPWTTYTTYTTTEKCNKE
TDKSKLQQCNTAVVVNVPSPLGNTPHGYKYACQCKIPTNEETCDDRKEY
MNQWSCGSARTMKRGYKNDNYELCKYNGVDVKPTTVRSNSSKLD.
SEQ ID NO. 2:
AHIVMVDAYKPTK.
SEQ ID NO. 3:
GGEYFTLQIRGRERFEEFREKNEALELKDAQAGKEPGG.
SEQ ID NO. 4:
AMVDTLSGLSSEQGQSGDMTIEEDSATHIKFSKRDEDGKELAGATMELR
DSSGKTISTWISDGQVKDFYLYPGKYTFVETAAPDGYEVATAITFTVNE
QGQVTVNGKATKGDAHI.

Preferably, the catenated VAR2CSA recombinant protein further includes a protein tag and a spatial linker.

Preferably, the protein tag includes any one or a combination of at least two of PNE-tag, human c-Myc-tag, CaptureSelect™ C-tag, FLAG-tag, 3×FLAG-tag, Twin-Strep-tag, Strep-tag, 6×His-tag, V5 tag, S-tag, HA-tag, VSV-G-tag, GST-tag, HaloTag, XTEN-tag or huEGFRt-tag.

Preferably, a polypeptide sequence of Twin-Strep-tag includes a sequence shown in SEQ ID NO. 5.

Preferably, the spatial linker includes a flexible linker and a helix-forming peptide linker.

Preferably, a polypeptide sequence of the flexible linker includes a sequence shown in SEQ ID NO. 6.

Preferably, a polypeptide sequence of the helix-forming peptide linker includes a sequence shown in SEQ ID NO. 7.

SEQ ID NO. 5:
SAWSHPQFEKGGGSGGGSGGSSAWSHPQFEK.
SEQ ID NO. 6:
GGGGS.
SEQ ID NO. 7:
LVGEAAAKEAAAKA.

In the present application, the binding domain, SpyTag, the p53dim domain, SpyCatcher and the protein tag are randomly arranged and linked via the spatial linker to form catenated VAR2CSA recombinant proteins with similar functions but different sequences and structures.

Preferably, the flexible linker is located between SpyTag and the p53dim domain.

Preferably, the helix-forming peptide linker is located between SpyCatcher and Twin-Strep-tag.

Preferably, a polypeptide sequence of the catenated VAR2CSA recombinant protein includes a sequence shown in SEQ ID NO. 8, SEQ ID NO. 9 or SEQ ID NO. 10.

SEQ ID NO. 8:
MAHIVMVDAYKPTKGGGGSGGEYFTLQIRGRERFEEFREKNEALELKDAQAGKEPGG
VDNYIKGDPYFAEYATKLSFILNSSDANNPSEKIQKNNDEVCNCNESGIASVEQEQISDPS
SNKTCITHSSIKANKKKVCKHVKLGVRENDKDLRVCVIEHTSLSGVENCCCQDFLRILQ
ENCSDNKSGSSSNGSCNNKNQEACEKNLEKVLASLTNCYKCDKCKSEQSKKNNKNWI
WKKSSGKEGGLQKEYANTIGLPPRTQSLCLVVCLDEKGKKTQELKNIRTNSELLKEWII
AAFHEGKNLKPSHEKKNDDNGKKLCKALEYSFADYGDLIKGTSIWDNEYTKDLELNL
QKIFGKLFRKYIKKNNTAEQDTSYSSLDELRESWWNTNKKYIWLAMKHGAGMNSTTC
CGDGSVTGSGSSCDDIPTIDLIPQYLRFLQEWVEHFCKQRQEKVKPVIENCKSCKESGGT
CNGECKTECKNKCEVYKKFIEDCKGGDGTAGSSWVKRWDQIYKRYSKYIEDAKRNRK
AGTKNCGPSSTTNAAENKCVQSDIDSFFKHLIDIGLTTPSSYLSIVLDDNICGADKAPWT
TYTTYTTTEKCNKETDKSKLQQCNTAVVVNVPSPLGNTPHGYKYACQCKIPTNEETCD
DRKEYMNQWSCGSARTMKRGYKNDNYELCKYNGVDVKPTTVRSNSSKLDGTAMVD
TLSGLSSEQGQSGDMTIEEDSATHIKFSKRDEDGKELAGATMELRDSSGKTISTWISDGQ
VKDFYLYPGKYTFVETAAPDGYEVATAITFTVNEQGQVTVNGKATKGDAHI.
SEQ ID NO. 9:
MNYIKGDPYFAEYATKLSFILNSSDANNPSEKIQKNNDEVCNCNESGIASVEQEQISDPSS
NKTCITHSSIKANKKKVCKHVKLGVRENDKDLRVCVIEHTSLSGVENCCCQDFLRILQE
NCSDNKSGSSSNGSCNNKNQEACEKNLEKVLASLTNCYKCDKCKSEQSKKNNKNWIW
KKSSGKEGGLQKEYANTIGLPPRTQSLCLVVCLDEKGKKTQELKNIRTNSELLKEWIIAA
FHEGKNLKPSHEKKNDDNGKKLCKALEYSFADYGDLIKGTSIWDNEYTKDLELNLQKI
FGKLFRKYIKKNNTAEQDTSYSSLDELRESWWNTNKKYIWLAMKHGAGMNSTTCCGD
GSVTGSGSSCDDIPTIDLIPQYLRFLQEWVEHFCKQRQEKVKPVIENCKSCKESGGTCNG
ECKTECKNKCEVYKKFIEDCKGGDGTAGSSWVKRWDQIYKRYSKYIEDAKRNRKAGT
KNCGPSSTTNAAENKCVQSDIDSFFKHLIDIGLTTPSSYLSIVLDDNICGADKAPWTTYT
TYTTTEKCNKETDKSKLQQCNTAVVVNVPSPLGNTPHGYKYACQCKIPTNEETCDDRK
EYMNQWSCGSARTMKRGYKNDNYELCKYNGVDVKPTTVRSNSSKLDGSAHIVMVDA
YKPTKGGGGSGGEYFTLQIRGRERFEEFREKNEALELKDAQAGKEPGGVDNYIKGDPY
FAEYATKLSFILNSSDANNPSEKIQKNNDEVCNCNESGIASVEQEQISDPSSNKTCITHSSI
KANKKKVCKHVKLGVRENDKDLRVCVIEHTSLSGVENCCCQDFLRILQENCSDNKSGS
SSNGSCNNKNQEACEKNLEKVLASLTNCYKCDKCKSEQSKKNNKNWIWKKSSGKEGG
LQKEYANTIGLPPRTQSLCLVVCLDEKGKKTQELKNIRTNSELLKEWIIAAFHEGKNLKP
SHEKKNDDNGKKLCKALEYSFADYGDLIKGTSIWDNEYTKDLELNLQKIFGKLFRKYI
KKNNTAEQDTSYSSLDELRESWWNTNKKYIWLAMKHGAGMNSTTCCGDGSVTGSGS
SCDDIPTIDLIPQYLRFLQEWVEHFCKQRQEKVKPVIENCKSCKESGGTCNGECKTECK
NKCEVYKKFIEDCKGGDGTAGSSWVKRWDQIYKRYSKYIEDAKRNRKAGTKNCGPSS
TTNAAENKCVQSDIDSFFKHLIDIGLTTPSSYLSIVLDDNICGADKAPWTTYTTYTTTEK
CNKETDKSKLQQCNTAVVVNVPSPLGNTPHGYKYACQCKIPTNEETCDDRKEYMNQW
SCGSARTMKRGYKNDNYELCKYNGVDVKPTTVRSNSSKLDGTAMVDTLSGLSSEQGQ
SGDMTIEEDSATHIKFSKRDEDGKELAGATMELRDSSGKTISTWISDGQVKDFYLYPGK
YTFVETAAPDGYEVATAITFTVNEQGQVTVNGKATKGDAHI.
SEQ ID NO. 10:
MAHIVMVDAYKPTKNYIKGDPYFAEYATKLSFILNSSDANNPSEKIQKNNDEVCNCNES
GIASVEQEQISDPSSNKTCITHSSIKANKKKVCKHVKLGVRENDKDLRVCVIEHTSLSGV
ENCCCQDFLRILQENCSDNKSGSSSNGSCNNKNQEACEKNLEKVLASLTNCYKCDKCK
SEQSKKNNKNWIWKKSSGKEGGLQKEYANTIGLPPRTQSLCLVVCLDEKGKKTQELKN
IRTNSELLKEWIIAAFHEGKNLKPSHEKKNDDNGKKLCKALEYSFADYGDLIKGTSIWD
NEYTKDLELNLQKIFGKLFRKYIKKNNTAEQDTSYSSLDELRESWWNTNKKYIWLAMK
HGAGMNSTTCCGDGSVTGSGSSCDDIPTIDLIPQYLRFLQEWVEHFCKQRQEKVKPVIE
NCKSCKESGGTCNGECKTECKNKCEVYKKFIEDCKGGDGTAGSSWVKRWDQIYKRYS
KYIEDAKRNRKAGTKNCGPSSTTNAAENKCVQSDIDSFFKHLIDIGLTTPSSYLSIVLDD
NICGADKAPWTTYTTYTTTEKCNKETDKSKLQQCNTAVVVNVPSPLGNTPHGYKYACQ
CKIPTNEETCDDRKEYMNQWSCGSARTMKRGYKNDNYELCKYNGVDVKPTTVRSNSS
KLDGSGGEYFTLQIRGRERFEEFREKNEALELKDAQAGKEPGGVDNYIKGDPYFAEYAT
KLSFILNSSDANNPSEKIQKNNDEVCNCNESGIASVEQEQISDPSSNKTCITHSSIKANKK
KVCKHVKLGVRENDKDLRVCVIEHTSLSGVENCCCQDFLRILQENCSDNKSGSSSNGSC
NNKNQEACEKNLEKVLASLTNCYKCDKCKSEQSKKNNKNWIWKKSSGKEGGLQKEY
ANTIGLPPRTQSLCLVVCLDEKGKKTQELKNIRTNSELLKEWIIAAFHEGKNLKPSHEKK
NDDNGKKLCKALEYSFADYGDLIKGTSIWDNEYTKDLELNLQKIFGKLFRKYIKKNNT
AEQDTSYSSLDELRESWWNTNKKYIWLAMKHGAGMNSTTCCGDGSVTGSGSSCDDIP
TIDLIPQYLRFLQEWVEHFCKQRQEKVKPVIENCKSCKESGGTCNGECKTECKNKCEVY
KKFIEDCKGGDGTAGSSWVKRWDQIYKRYSKYIEDAKRNRKAGTKNCGPSSTTNAAE
NKCVQSDIDSFFKHLIDIGLTTPSSYLSIVLDDNICGADKAPWTTYTTYTTTEKCNKETD
KSKLQQCNTAVVVNVPSPLGNTPHGYKYACQCKIPTNEETCDDRKEYMNQWSCGSAR
TMKRGYKNDNYELCKYNGVDVKPTTVRSNSSKLDGTAMVDTLSGLSSEQGQSGDMTI
EEDSATHIKFSKRDEDGKELAGATMELRDSSGKTISTWISDGQVKDFYLYPGKYTFVET
AAPDGYEVATAITFTVNEQGQVTVNGKATKGDAHI.

In a second aspect, the present application provides a method for preparing the catenated VAR2CSA recombinant protein described in the first aspect. The preparation method includes the following steps:
constructing an expression vector including a coding gene of the catenated VAR2CSA recombinant protein described in the first aspect, transfecting the expression vector into cells, culturing the cells, and performing protein purification to obtain the catenated VAR2CSA recombinant protein.

In a third aspect, the present application provides a nucleic acid molecule. The nucleic acid molecule includes a coding gene of the catenated VAR2CSA recombinant protein described in the first aspect.

Preferably, the nucleic acid molecule includes a deoxyribonucleic acid sequence shown in SEQ ID NO. 11, SEQ ID NO. 12 or SEQ ID NO. 13 or a variant thereof having at least 80% nucleotide identity or more.

SEQ ID NO. 11:
atggcgcacattgttatggtggacgcgtacaaaccgaccaagggtggcggtggcagcggtggcgagtatttcaccctgcagatccgtgg
ccgtgaacgtttcgaggaatttcgtgagaaaaacgaagcgctggagctgaaagatgcgcaagcgggcaaggagccgggtggcgtcga
caactacatcaaaggcgatccgtatttcgcggaatacgcgaccaagctgagctttattctgaacagcagcgacgcgaacaacccgagcg
agaaaatccagaagaacaacgatgaagtgtgcaactgcaacgagagcggtattgcgagcgttgagcaggaacaaatcagcgacccga
gcagcaacaaaacctgcattacccacagcagcatcaaggcgaacaagaaaaaggtttgcaaacacgtgaagctgggcgttcgtgagaa
cgacaaggatctgcgtgtttgcgtgattgagcacaccagcctgagcggtgtggaaaactgctgctgccaggactttctgcgtatcctgcaa
gaaaactgcagcgataacaaaagcggtagcagcagcaacggcagctgcaacaacaagaaccaggaagcgtgcgagaaaaacctgga
gaaggttctggcgagcctgaccaactgctacaaatgcgacaaatgcaagagcgaacaaagcaaaaagaacaacaagaactggatttgg
aaaaagagcagcggcaaagaaggtggcctgcagaaggagtatgcgaacaccatcggtctgccgccgcgtacccaaagcctgtgcctg
gtggtttgcctggatgaaaaaggtaaaaagacccaggagctgaagaacatccgtaccaacagcgaactgctgaaagagtggatcattgc
ggcgttccacgagggcaaaaacctgaagccgagccacgagaagaagaacgacgataacggtaaaaagctgtgcaaggcgctggagt
atagctttgcggactacggtgatctgattaaaggcaccagcatctgggacaacgaatacaccaaggatctggagctgaacctgcagaaaa
ttttcggcaagctgttccgtaagtacatcaaaaagaacaacaccgcggaacaagacaccagctatagcagcctggatgaactgcgtgagt
cctggtggaacaccaacaaaaagtacatctggctggcgatgaaacacggtgcgggcatgaacagcaccacctgctgcggtgatggcag
cgtgaccggtagcggcagcagctgcgacgatatcccgaccattgatctgatcccgcagtatctgcgtttcctgcaagaatgggttgagcac
ttttgcaaacagcgtcaagaaaaagttaagccggtgattgagaactgcaaaagctgcaaggaaagcggtggcacctgcaacggtgaatg
caagaccgagtgcaaaaacaagtgcgaggtgtacaaaaagttcatcgaagactgcaaaggtggcgatggcaccgcgggcagcagctg
ggttaagcgttgggaccagatttacaagcgttatagcaaatacatcgaggatgcgaagcgtaaccgtaaagcgggcaccaagaactgcg
gtccgagcagcaccaccaacgcggcggaaaacaaatgcgtgcaaagcgacattgatagcttctttaagcacctgatcgacattggcctga
ccaccccgagcagctacctgagcattgttctggacgataacatttgcggtgcggacaaggcgccgtggaccacctataccacctacacca
ccaccgaaaaatgcaacaaggaaaccgataaaagcaagctgcagcaatgcaacaccgcggtggttgtgaacgttccgagcccgctggg
taacaccccgcacggctacaaatatgcgtgccagtgcaagatcccgaccaacgaggaaacctgcgacgatcgtaaagagtatatgaacc
aatggagctgcggtagcgcgcgtaccatgaaacgtggctataagaacgacaactacgaactgtgcaaatataacggcgttgatgtgaagc
cgaccaccgtgcgtagcaacagcagcaagctggacggtaccgcgatggttgataccctgagcggtctgagcagcgagcagggtcaaa
gcggcgacatgaccattgaggaagatagcgcgacccacatcaaattcagcaagcgtgacgaagatggtaaagagctggcgggcgcga
ccatggaactgcgtgacagcagcggcaagaccattagcacctggatcagcgacggccaggtgaaagatttctacctgtatccgggcaag
tacacctttgttgaaaccgcggcgccggatggttatgaagtggcgaccgcgattacctttaccgttaacgagcagggtcaagttaccgtga
acggtaaagcgaccaagggcgatgcgcacatctaa.
SEQ ID NO. 12:
atgaactacatcaaaggcgatccgtatttcgcggaatacgcgaccaagctgagctttattctgaacagcagcgacgcgaacaacccgagc
gagaaaatccagaagaacaacgatgaagtgtgcaactgcaacgagagcggtattgcgagcgttgagcaggaacaaatcagcgacccg
agcagcaacaaaacctgcattacccacagcagcatcaaggcgaacaagaaaaaggtttgcaaacacgtgaagctgggcgttcgtgaga
acgacaaggatctgcgtgtttgcgtgattgagcacaccagcctgagcggtgtggaaaactgctgctgccaggactttctgcgtatcctgca
agaaaactgcagcgataacaaaagcggtagcagcagcaacggcagctgcaacaacaagaaccaggaagcgtgcgagaaaaacctgg
agaaggttctggcgagcctgaccaactgctacaaatgcgacaaatgcaagagcgaacaaagcaaaaagaacaacaagaactggatttg
gaaaaagagcagcggcaaagaaggtggcctgcagaaggagtatgcgaacaccatcggtctgccgccgcgtacccaaagcctgtgcct
ggtggtttgcctggatgaaaaaggtaaaaagacccaggagctgaagaacatccgtaccaacagcgaactgctgaaagagtggatcattg
cggcgttccacgagggcaaaaacctgaagccgagccacgagaagaagaacgacgataacggtaaaaagctgtgcaaggcgctggag
tatagctttgcggactacggtgatctgattaaaggcaccagcatctgggacaacgaatacaccaaggatctggagctgaacctgcagaaa
attttcggcaagctgttccgtaagtacatcaaaaagaacaacaccgcggaacaagacaccagctatagcagcctggatgaactgcgtgag
tcctggtggaacaccaacaaaaagtacatctggctggcgatgaaacacggtgcgggcatgaacagcaccacctgctgcggtgatggca
gcgtgaccggtagcggcagcagctgcgacgatatcccgaccattgatctgatcccgcagtatctgcgtttcctgcaagaatgggttgagca
cttttgcaaacagcgtcaagaaaaagttaagccggtgattgagaactgcaaaagctgcaaggaaagcggtggcacctgcaacggtgaat
gcaagaccgagtgcaaaaacaagtgcgaggtgtacaaaaagttcatcgaagactgcaaaggtggcgatggcaccgcgggcagcagct
gggttaagcgttgggaccagatttacaagcgttatagcaaatacatcgaggatgcgaagcgtaaccgtaaagcgggcaccaagaactgc
ggtccgagcagcaccaccaacgcggcggaaaacaaatgcgtgcaaagcgacattgatagcttctttaagcacctgatcgacattggcctg
accaccccgagcagctacctgagcattgttctggacgataacatttgcggtgcggacaaggcgccgtggaccacctataccacctacacc
accaccgaaaaatgcaacaaggaaaccgataaaagcaagctgcagcaatgcaacaccgcggtggttgtgaacgttccgagcccgctgg
gtaacaccccgcacggctacaaatatgcgtgccagtgcaagatcccgaccaacgaggaaacctgcgacgatcgtaaagagtatatgaac
caatggagctgcggtagcgcgcgtaccatgaaacgtggctataagaacgacaactacgaactgtgcaaatataacggcgttgatgtgaag
ccgaccaccgtgcgtagcaacagcagcaagctggacggatccgcgcacattgttatggtggacgcgtacaaaccgaccaagggtggc
ggtggcagcggtggcgagtatttcaccctgcagatccgtggccgtgaacgtttcgaggaatttcgtgagaaaaacgaagcgctggagctg
aaagatgcgcaagcgggcaaggagccgggggcgtcgacaactacatcaaaggcgatccgtatttcgcggaatacgcgaccaagctg
agctttattctgaacagcagcgacgcgaacaacccgagcgagaaaatccagaagaacaacgatgaagtgtgcaactgcaacgagagcg
gtattgcgagcgttgagcaggaacaaatcagcgacccgagcagcaacaaaacctgcattacccacagcagcatcaaggcgaacaagaa
aaaggtttgcaaacacgtgaagctgggcgttcgtgagaacgacaaggatctgcgtgtttgcgtgattgagcacaccagcctgagcggtgt
ggaaaactgctgctgccaggactttctgcgtatcctgcaagaaaactgcagcgataacaaaagcggtagcagcagcaacggcagctgca
acaacaagaaccaggaagcgtgcgagaaaaacctggagaaggttctggcgagcctgaccaactgctacaaatgcgacaaatgcaaga
gcgaacaaagcaaaaagaacaacaagaactggatttggaaaaagagcagcggcaaagaaggtggcctgcagaaggagtatgcgaac
accatcggtctgccgccgcgtacccaaagcctgtgcctggtggtttgcctggatgaaaaaggtaaaaagacccaggagctgaagaacat
ccgtaccaacagcgaactgctgaaagagtggatcattgcggcgttccacgagggcaaaaacctgaagccgagccacgagaagaagaa
cgacgataacggtaaaaagctgtgcaaggcgctggagtatagctttgcggactacggtgatctgattaaaggcaccagcatctgggacaa
cgaatacaccaaggatctggagctgaacctgcagaaaattttcggcaagctgttccgtaagtacatcaaaaagaacaacaccgcggaaca
agacaccagctatagcagcctggatgaactgcgtgagtcctggtggaacaccaacaaaaagtacatctggctggcgatgaaacacggtg
cgggcatgaacagcaccacctgctgcggtgatggcagcgtgaccggtagcggcagcagctgcgacgatatcccgaccattgatctgatc
ccgcagtatctgcgtttcctgcaagaatgggttgagcacttttgcaaacagcgtcaagaaaaagttaagccggtgattgagaactgcaaaa
gctgcaaggaaagcggtggcacctgcaacggtgaatgcaagaccgagtgcaaaaacaagtgcgaggtgtacaaaaagttcatcgaaga
ctgcaaaggtggcgatggcaccgcgggcagcagctgggttaagcgttgggaccagatttacaagcgttatagcaaatacatcgaggatg
cgaagcgtaaccgtaaagcgggcaccaagaactgcggtccgagcagcaccaccaacgcggcggaaaacaaatgcgtgcaaagcgac
attgatagcttctttaagcacctgatcgacattggcctgaccaccccgagcagctacctgagcattgttctggacgataacatttgcggtgcg
gacaaggcgccgtggaccacctataccacctacaccaccaccgaaaaatgcaacaaggaaaccgataaaagcaagctgcagcaatgc
aacaccgcggtggttgtgaacgttccgagcccgctgggtaacaccccgcacggctacaaatatgcgtgccagtgcaagatcccgaccaa
cgaggaaacctgcgacgatcgtaaagagtatatgaaccaatggagctgcggtagcgcgcgtaccatgaaacgtggctataagaacgac
aactacgaactgtgcaaatataacggcgttgatgtgaagccgaccaccgtgcgtagcaacagcagcaagctggacggtaccgcgatggt
tgataccctgagcggtctgagcagcgagcagggtcaaagcggcgacatgaccattgaggaagatagcgcgacccacatcaaattcagc
aagcgtgacgaagatggtaaagagctggcgggcgcgaccatggaactgcgtgacagcagcggcaagaccattagcacctggatcagc
gacggccaggtgaaagatttctacctgtatccgggcaagtacacctttgttgaaaccgcggcgccggatggttatgaagtggcgaccgcg
attacctttaccgttaacgagcagggtcaagttaccgtgaacggtaaagcgaccaagggcgatgcgcacatctaa.
SEQ ID NO. 13:
atggcgcacattgttatggtggacgcgtacaaaccgaccaagaactacatcaaaggcgatccgtatttcgcggaatacgcgaccaagctg
agctttattctgaacagcagcgacgcgaacaacccgagcgagaaaatccagaagaacaacgatgaagtgtgcaactgcaacgagagcg
gtattgcgagcgttgagcaggaacaaatcagcgacccgagcagcaacaaaacctgcattacccacagcagcatcaaggcgaacaagaa
aaaggtttgcaaacacgtgaagctgggcgttcgtgagaacgacaaggatctgcgtgtttgcgtgattgagcacaccagcctgagcggtgt
ggaaaactgctgctgccaggactttctgcgtatcctgcaagaaaactgcagcgataacaaaagcggtagcagcagcaacggcagctgca
acaacaagaaccaggaagcgtgcgagaaaaacctggagaaggttctggcgagcctgaccaactgctacaaatgcgacaaatgcaaga
gcgaacaaagcaaaaagaacaacaagaactggatttggaaaaagagcagcggcaaagaaggtggcctgcagaaggagtatgcgaac
accatcggtctgccgccgcgtacccaaagcctgtgcctggtggtttgcctggatgaaaaaggtaaaaagacccaggagctgaagaacat
ccgtaccaacagcgaactgctgaaagagtggatcattgcggcgttccacgagggcaaaaacctgaagccgagccacgagaagaagaa
cgacgataacggtaaaaagctgtgcaaggcgctggagtatagctttgcggactacggtgatctgattaaaggcaccagcatctgggacaa
cgaatacaccaaggatctggagctgaacctgcagaaaattttcggcaagctgttccgtaagtacatcaaaaagaacaacaccgcggaaca
agacaccagctatagcagcctggatgaactgcgtgagtcctggtggaacaccaacaaaaagtacatctggctggcgatgaaacacggtg
cgggcatgaacagcaccacctgctgcggtgatggcagcgtgaccggtagcggcagcagctgcgacgatatcccgaccattgatctgatc
ccgcagtatctgcgtttcctgcaagaatgggttgagcacttttgcaaacagcgtcaagaaaaagttaagccggtgattgagaactgcaaaa
gctgcaaggaaagcggtggcacctgcaacggtgaatgcaagaccgagtgcaaaaacaagtgcgaggtgtacaaaaagttcatcgaaga
ctgcaaaggtggcgatggcaccgcgggcagcagctgggttaagcgttgggaccagatttacaagcgttatagcaaatacatcgaggatg
cgaagcgtaaccgtaaagcgggcaccaagaactgcggtccgagcagcaccaccaacgcggcggaaaacaaatgcgtgcaaagcgac
attgatagcttctttaagcacctgatcgacattggcctgaccaccccgagcagctacctgagcattgttctggacgataacatttgcggtgcg
gacaaggcgccgtggaccacctataccacctacaccaccaccgaaaaatgcaacaaggaaaccgataaaagcaagctgcagcaatgc
aacaccgcggtggttgtgaacgttccgagcccgctgggtaacaccccgcacggctacaaatatgcgtgccagtgcaagatcccgaccaa
cgaggaaacctgcgacgatcgtaaagagtatatgaaccaatggagctgcggtagcgcgcgtaccatgaaacgtggctataagaacgac
aactacgaactgtgcaaatataacggcgttgatgtgaagccgaccaccgtgcgtagcaacagcagcaagctggacggatccggtggcg
agtatttcaccctgcagatccgtggccgtgaacgtttcgaggaatttcgtgagaaaaacgaagcgctggagctgaaagatgcgcaagcgg
gcaaggagccgggtggcgtcgacaactacatcaaaggcgatccgtatttcgcggaatacgcgaccaagctgagctttattctgaacagca
gcgacgcgaacaacccgagcgagaaaatccagaagaacaacgatgaagtgtgcaactgcaacgagagcggtattgcgagcgttgagc
aggaacaaatcagcgacccgagcagcaacaaaacctgcattacccacagcagcatcaaggcgaacaagaaaaaggtttgcaaacacgt
gaagctgggcgttcgtgagaacgacaaggatctgcgtgtttgcgtgattgagcacaccagcctgagcggtgtggaaaactgctgctgcca
ggactttctgcgtatcctgcaagaaaactgcagcgataacaaaagcggtagcagcagcaacggcagctgcaacaacaagaaccaggaa
gcgtgcgagaaaaacctggagaaggttctggcgagcctgaccaactgctacaaatgcgacaaatgcaagagcgaacaaagcaaaaag
aacaacaagaactggatttggaaaaagagcagcggcaaagaaggtggcctgcagaaggagtatgcgaacaccatcggtctgccgccg
cgtacccaaagcctgtgcctggtggtttgcctggatgaaaaaggtaaaaagacccaggagctgaagaacatccgtaccaacagcgaact
gctgaaagagtggatcattgcggcgttccacgagggcaaaaacctgaagccgagccacgagaagaagaacgacgataacggtaaaaa
gctgtgcaaggcgctggagtatagctttgcggactacggtgatctgattaaaggcaccagcatctgggacaacgaatacaccaaggatct
ggagctgaacctgcagaaaattttcggcaagctgttccgtaagtacatcaaaaagaacaacaccgcggaacaagacaccagctatagca
gcctggatgaactgcgtgagtcctggtggaacaccaacaaaaagtacatctggctggcgatgaaacacggtgcgggcatgaacagcac
cacctgctgcggtgatggcagcgtgaccggtagcggcagcagctgcgacgatatcccgaccattgatctgatcccgcagtatctgcgtttc
ctgcaagaatgggttgagcacttttgcaaacagcgtcaagaaaaagttaagccggtgattgagaactgcaaaagctgcaaggaaagcggt
ggcacctgcaacggtgaatgcaagaccgagtgcaaaaacaagtgcgaggtgtacaaaaagttcatcgaagactgcaaaggtggcgatg
gcaccgcgggcagcagctgggttaagcgttgggaccagatttacaagcgttatagcaaatacatcgaggatgcgaagcgtaaccgtaaa
gcgggcaccaagaactgcggtccgagcagcaccaccaacgcggcggaaaacaaatgcgtgcaaagcgacattgatagcttctttaagc
acctgatcgacattggcctgaccaccccgagcagctacctgagcattgttctggacgataacatttgcggtgcggacaaggcgccgtgga
ccacctataccacctacaccaccaccgaaaaatgcaacaaggaaaccgataaaagcaagctgcagcaatgcaacaccgcggtggttgt
gaacgttccgagcccgctgggtaacaccccgcacggctacaaatatgcgtgccagtgcaagatcccgaccaacgaggaaacctgcgac
gatcgtaaagagtatatgaaccaatggagctgcggtagcgcgcgtaccatgaaacgtggctataagaacgacaactacgaactgtgcaa
atataacggcgttgatgtgaagccgaccaccgtgcgtagcaacagcagcaagctggacggtaccgcgatggttgataccctgagcggtc
tgagcagcgagcagggtcaaagcggcgacatgaccattgaggaagatagcgcgacccacatcaaattcagcaagcgtgacgaagatg
gtaaagagctggcgggcgcgaccatggaactgcgtgacagcagcggcaagaccattagcacctggatcagcgacggccaggtgaaa
gatttctacctgtatccgggcaagtacacctttgttgaaaccgcggcgccggatggttatgaagtggcgaccgcgattacctttaccgttaac
gagcagggtcaagttaccgtgaacggtaaagcgaccaagggcgatgcgcacatctaa.

In a fourth aspect, the present application provides a chimeric antigen receptor cell system. The chimeric antigen receptor cell system includes the catenated VAR2CSA recombinant protein described in the first aspect and a chimeric antigen receptor cell. The chimeric antigen receptor cell expresses a chimeric antigen receptor that recognizes the catenated VAR2CSA recombinant protein.

In the chimeric antigen receptor cell system of the present application, the chimeric antigen receptor cell can recognize and bind to the catenated VAR2CSA recombinant protein and thus possesses the ability to recognize and bind to the tumor-specific antigen pl-CSA, that is, the chimeric antigen receptor cell uses the catenated VAR2CSA recombinant protein as the navigation system, and after entering the body, the chimeric antigen receptor cell and the catenated VAR2CSA recombinant protein can automatically be “assembled”, capture and kill the tumor cells. Since the catenated VAR2CSA recombinant protein has higher stability and affinity, the anti-tumor activity of the chimeric antigen receptor cell is enabled to become stronger and more durable. Furthermore, since the catenated VAR2CSA recombinant protein is free from the chimeric antigen receptor cell, the catenated VAR2CSA recombinant protein is also a “safety switch” of the chimeric antigen receptor cell in addition to the navigation system, and the function of the chimeric antigen receptor cell can be regulated indirectly by regulating the content of the catenated VAR2CSA recombinant protein in the system. In the event of serious toxic side effects such as cytokine release syndrome (CRS) or immune effector cell-associated neurotoxicity syndrome (ICANS), the chimeric antigen receptor cell which is dependent on the catenated VAR2CSA recombinant protein to function can be inactivated by cutting off the supply of the catenated VAR2CSA recombinant protein in the system, thereby enabling the system to be controllable and enhancing the safety of the system.

Preferably, the cell is an immune effector cell, and preferably is any one or a combination of at least two of T cells, B cells, NK cells, NKT cells, dendritic cells or macrophages.

In the present application, any other chimeric antigen receptor cell capable of directly or indirectly recognizing the catenated VAR2CSA recombinant protein falls within the protection scope of the present application when it also achieves the effect described in the present application.

Preferably, the chimeric antigen receptor includes a domain recognizing the catenated VAR2CSA recombinant protein.

Preferably, the chimeric antigen receptor further includes a hinge region, a transmembrane domain and an intracellular co-stimulatory signaling domain.

Preferably, the domain recognizing the catenated VAR2CSA recombinant protein includes a single-chain fragment variable antibody composed of a heavy chain variable region and a light chain variable region.

Preferably, the gene coding the heavy chain variable region of the single-chain fragment variable antibody is a deoxyribonucleic acid sequence shown in SEQ ID NO. 14 or a variant thereof having at least 80% nucleotide identity or more.

Preferably, the gene coding the light chain variable region of the single-chain fragment variable antibody is a deoxyribonucleic acid sequence shown in SEQ ID NO. 15 or a variant thereof having at least 80% nucleotide identity or more.

SEQ ID NO. 14:
gaggtgaagctggtggaaagcggcggcggactggtgaaacctggaggca
gcctgaagctcagctgcgccgctagcggatttacatttagcaactacgc
catgagctgggtgcggcagagccccgagaggcgcctggaatgggtcgct
gagatcagcatcaccggcagatacacctactaccccgacaccgttacag
gccggttcaccatcagccgggacaacgccaagaacaccctgtacctgga
gatgagttctctgagatctgaagataccgccatgtactactgcaccaga
gagggctacgactacgccccttcttggttcgcctactggggacagggaa
ccctggtcaccgtgtccgcc.
SEQ ID NO. 15:
gacgtggtgatgacccagacacctctgagcctgcctgtgtctctgggcg
accaggccagcatcagctgtagaagcggccagaccctggtgcacagaaa
tggcatcacatacctggaatggtatctgcagaagcctggccaatctcca
aagctgctgatctacaaggtgtccaaccggttcagcggcgtgcccgata
gattcagcggcagcggctccggcaccgacttcaccctgaaaatctccag
agtggaagccgaggatctgggaatctactattgcttccagggctctcac
gtgcctagaacattcggcggaggtacaaagctggagattaag.

Preferably, the hinge region is a human CD8α hinge region.

Preferably, the transmembrane domain is a human CD28 transmembrane domain.

Preferably, the intracellular signaling domain is any one or a combination of at least two of a human CD27 intracellular signaling domain, a human CD134 intracellular signaling domain, a human CD28 intracellular signaling domain or a human 4-1BB intracellular signaling domain.

Preferably, the amino-terminus of the chimeric antigen receptor includes a CD8α signal peptide.

Preferably, the carboxyl-terminus of the chimeric antigen receptor includes a human CD3ζ intracellular signaling domain.

Preferably, the chimeric antigen receptor includes a CD8α signal peptide, a single-chain fragment variable antibody recognizing the catenated VAR2CSA recombinant protein, a human CD8α hinge region, a human CD28 transmembrane domain, a human CD28 intracellular signaling domain, a human 4-1BB intracellular signaling domain and a human CD3ζ intracellular signaling domain, and these multiple functional domains are in tandem sequentially.

Preferably, the chimeric antigen receptor includes a polypeptide sequence shown in SEQ ID NO. 16.

SEQ ID NO. 16:
MALPVTALLLPLALLLHAARPDVVMTQTPLSLPVSLGDQASISCRSGQT
LVHRNGITYLEWYLQKPGQSPKLLIYKVSNRFSGVPDRFSGSGSGTDFT
LKISRVEAEDLGIYYCFQGSHVPRTFGGGTKLEIKGGGGSGGGGSGGGG
SEVKLVESGGGLVKPGGSLKLSCAASGFTFSNYAMSWVRQSPERRLEWV
AEISITGRYTYYPDTVTGRFTISRDNAKNTLYLEMSSLRSEDTAMYYCT
REGYDYAPSWFAYWGQGTLVTVSATTTPAPRPPTPAPTIASQPLSLRPE
ACRPAAGGAVHTRGLDFACDFWVLVVVGGVLACYSLLVTVAFIIFWVRS
KRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDK
MAEAYSEIGMKGERRRGKGHDGLYQGLSTATKDTYDALHMQALPPR.

In a fifth aspect, the present application provides a chimeric antigen receptor cell. The chimeric antigen receptor cell expresses the catenated VAR2CSA recombinant protein described in the first aspect and a chimeric antigen receptor that recognizes the catenated VAR2CSA recombinant protein.

In a sixth aspect, the present application provides a pharmaceutical composition. The pharmaceutical composition includes any one or a combination of at least two of the catenated VAR2CSA recombinant protein described in the first aspect, the nucleic acid molecule described in the third aspect, the chimeric antigen receptor cell system described in the fourth aspect or the chimeric antigen receptor cell described in the fifth aspect.

Preferably, the pharmaceutical composition further includes a pharmaceutically acceptable adjuvant.

In a seventh aspect, the present application provides a use of the catenated VAR2CSA recombinant protein described in the first aspect, the nucleic acid molecule described in the third aspect, the chimeric antigen receptor cell system described in the fourth aspect, the chimeric antigen receptor cell described in the fifth aspect or the pharmaceutical composition described in the sixth aspect in the preparation of a drug for treating a tumor.

Preferably, the tumor is a solid tumor and/or a hematological tumor.

The tumor may be any tumor tissue or cell that can be specifically identified and bound by a VAR2CSA protein or by a domain in a VAR2CSA protein that can bind to placental-like chondroitin sulfate A (pl-CSA); may be a human solid tumor cell line, for example, a lung cancer cell line including NCI-H460 (a large cell lung cancer cell line, ATCC #HTB177), NCI-H520 (a squamous cell lung cancer cell line, ATCC #HTB182) and A549 (a lung adenocarcinoma cell line, ATCC #CCL185); may be a human melanoma cell line, including MP38 (a uveal melanoma cell line, ATCC #CRL-3296); or may be a human hematological tumor cell line, including Raji (a B-cell lymphoma cell line, ATCC #CCL86) and K562 (a human chronic myelogenous leukemia cell line, ATCC #CCL-243), etc.

Compared to the existing art, the present application has the beneficial effects described below.
(1) The present application designs and successfully prepares a catenated VAR2CSA recombinant protein, thereby significantly improving the protein stability and protein affinity for the tumor-specific antigen placenta-like chondroitin sulfate A.
(2) Since the chimeric antigen receptor cell in the chimeric antigen receptor cell system of the present application uses the catenated VAR2CSA recombinant protein as the navigation system, the chimeric antigen receptor cell system has stronger and more durable anti-tumor activity and good in vivo tumor therapeutic effects, and the function of the chimeric antigen receptor cell can be regulated by regulating the content of the catenated VAR2CSA recombinant protein in the system, which indicates that the occurrence of toxic side effects such as cytokine release syndrome (CRS) or immune effector cell-associated neurotoxicity syndrome (ICANS) due to the activation of CAR-T cells can be avoided by regulating the administration dose of the navigator protein in the clinical treatment, thereby providing better safety.

### BRIEF DESCRIPTION OF DRAWINGS

FIG. 1 shows a gene expression vector of a catenated VAR2CSA recombinant protein.

FIG. 2 shows the anti-tumor principle of a CAR-T cell system using a catenated Plasmodium VAR2CSA recombinant protein as the navigation system.

FIG. 3 shows an intracellular synthesis route of a catenated VAR2CSA recombinant protein.

FIG. 4 shows a plasmid map of an inducible prokaryotic expression vector of a catenated VAR2CSA recombinant protein.

FIG. 5 shows the polyacrylamide gel electrophoresis detection results of VAR2CSA recombination proteins purified with Strep-Tactin resin.

FIG. 6 shows the polyacrylamide gel electrophoresis detection results of VAR2CSA recombination proteins purified with Strep-Tactin resin and anion-exchange resin.

FIG. 7 shows the module compositions of Anti-CD19 CAR and Anti-rVAR2 CAR.

FIG. 8 shows the proportions of positive T cells expressing CAR in the normal T cell group, the CD19-CAR T cell group and the Anti-rVAR2-CAR T cell group analyzed by flow cytometry.

FIG. 9 shows CAR expression detected by western-blot.

FIG. 10 shows the affinity detection results between AXVB and 5H4 mAb.

FIG. 11 shows the affinity detection results between rVAR2 and 5H4 mAb.

FIG. 12 shows the affinity detection results between AVXVB and 5H4 mAb.

FIG. 13 shows the affinity detection results between VAXVB and 5H4 mAb.

FIG. 14 shows the relative mean fluorescence intensity analysis of the targeted binding of AXVB and rVAR2 to different types of tumor cell lines.

FIG. 15 shows the comparative analysis of the binding proportions of AXVB and rVAR2 to Raji cells under different temperature and time processing conditions.

FIG. 16 shows the comparative analysis of the binding proportions of AXVB and rVAR2 to K562 cells under different temperature and time processing conditions.

FIG. 17 shows the comparative analysis of the relative residual activity of AXVB and rVAR2 proteins binding to Raji cells under different temperature and time processing conditions.

FIG. 18 shows the comparative analysis of the relative residual activity of AXVB and rVAR2 proteins binding to K562 cells under different temperature and time processing conditions.

FIG. 19 shows the expression of CD19 antigen molecules on the membrane surface of Raji, K562 and H460 cells analyzed by flow cytometry.

FIG. 20 shows the comparison of in vitro cytotoxicity of navigator proteins (AXVB and rVAR2) or CAR-T cell systems using the navigator protein as the navigation system and CD19-CAR T cells to Raji cells.

FIG. 21 shows the comparison of in vitro cytotoxicity of navigator proteins (AXVB and rVAR2) or CAR-T cell systems using the navigator protein as the navigation system and CD19-CAR T cells to K562 cells.

FIG. 22 shows the comparison of in vitro cytotoxicity of navigator proteins (AXVB and rVAR2) or CAR-T cell systems using the navigator protein as the navigation system and CD19-CAR T cells to H460 cells.

FIG. 23 shows the in vitro cytokine secretion level comparison between normal T cells, CD19-CAR T cells and CAR-T cell systems using AXVB or rVAR2 as the navigation system, with Raji cells as target cells.

FIG. 24 shows the in vitro cytokine secretion level comparison between normal T cells, CD19-CAR T cells and CAR-T cell systems using AXVB or rVAR2 as the navigation system, with K562 cells as target cells.

FIG. 25 shows the secretion level of IL-2 in AXVB-[switch]-CAR T cell systems regulated by the catenated VAR2CSA recombinant protein AXVB.

FIG. 26 shows the secretion level of TNF-α in AXVB-[switch]-CAR T cell systems regulated by the catenated VAR2CSA recombinant protein AXVB.

FIG. 27 shows the secretion level of IFN-γ in AXVB-[switch]-CAR T cell systems regulated by the catenated VAR2CSA recombinant protein AXVB.

FIG. 28 shows the anti-tumor activity of AXVB-[switch]-CAR T cell systems regulated by the catenated VAR2CSA recombinant protein AXVB.

FIG. 29 shows the schematic diagrams of experimental grouping and experimental design for CAR-T cell therapy in animal model.

FIG. 30 shows the in vivo imaging assay results of Raji cell tumor-bearing mice treated with CAR-T cell therapy, with the tumor burden shown as quantified firefly luciferase-catalyzed D-luciferin substrate luminescence signals, five mice per group.

FIG. 31 illustrates the survival curve of Raji cell tumor-bearing mice, where * indicates that p<0.05, ** indicates that p<0.01, and ns indicates that p>0.05.

FIG. 32 shows the in vivo imaging assay results of K562 cell tumor-bearing mice treated with CAR-T cell therapy, with the tumor burden shown as quantified firefly luciferase-catalyzed D-luciferin substrate luminescence signals, five mice per group.

FIG. 33 shows the survival curve of K562 cell tumor-bearing mice, where * indicates that p<0.05, ** indicates that p<0.01, and ns indicates that p>0.05.

FIG. 34 shows in vivo tumor metastases in Raji cell tumor-bearing mice in the PBS control group, where black arrows point to the sites of tumor metastases.

### DETAILED DESCRIPTION

To further elaborate on the technical means adopted and effects achieved in the present application, the present application is further described below in conjunction with examples and drawings. It is to be understood that the specific examples set forth below are intended to explain the present application and are not to limit the present application.

Experiments without specific techniques or conditions specified in the examples are conducted according to techniques or conditions described in the literature in the art or according to product instructions. The reagents or instruments used herein without manufacturers specified are conventional products commercially available from proper channels.

### Example 1

This example designed a catenated VAR2CSA recombinant protein and a chimeric antigen receptor T cell system.

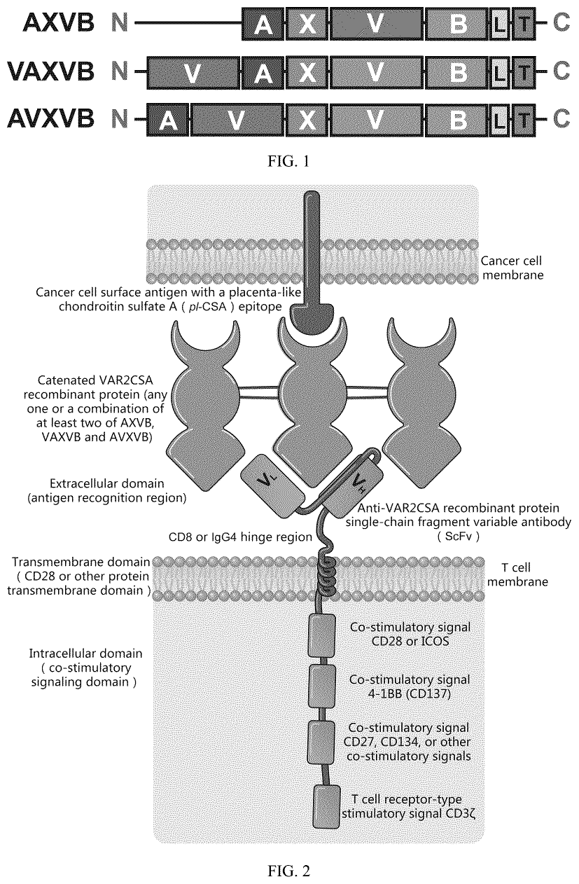

In this example, three catenated VAR2CSA recombinant proteins including a domain in the VAR2CSA protein that binds to placental-like chondroitin sulfate A (V), SpyTag (A), a p53dim domain (X), SpyCatcher (B), Twin-Strep-tag (T) and a helix-forming peptide linker (L) were designed, which were named AXVB, VAXVB and AVXVB, respectively. Their structural arrangement is illustrated in FIG. 1, and the polypeptide sequences are shown in SEQ ID NO. 8 (AXVB), SEQ ID NO. 9 (VAXVB) and SEQ ID NO. 10 (AVXVB), respectively.

In the chimeric antigen receptor cell system in this example, the chimeric antigen receptor cell was a chimeric antigen receptor T (CAR-T) cell, and the polypeptide sequence of the chimeric antigen receptor expressed by the chimeric antigen receptor cell was shown in SEQ ID NO. 16. The anti-tumor principle of the CAR-T cell system is illustrated in FIG. 2. The CAR-T cell could recognize and bind to the catenated VAR2CSA recombinant protein and in turn could recognize tumor tissue through interacting with pl-CSA and exert a killing effect.

### Example 2

In this example, the catenated VAR2CSA recombinant proteins (AXVB, VAXVB and AVXVB) described in Example 1 and the wild-type VAR2CSA recombinant protein (rVAR2, whose polypeptide sequences of the protein domains, except for the protein tag, were identical to the polypeptide sequences of the relevant domains of a wild-type Plasmodium VAR2CSA recombinant protein, and which did not have intra- or inter-protein molecular coupling polymers) were prepared.

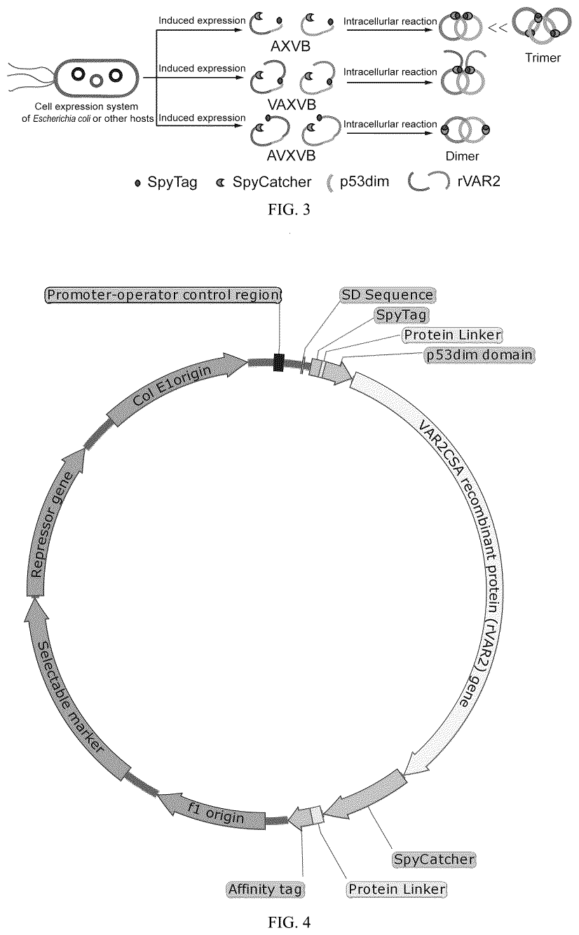

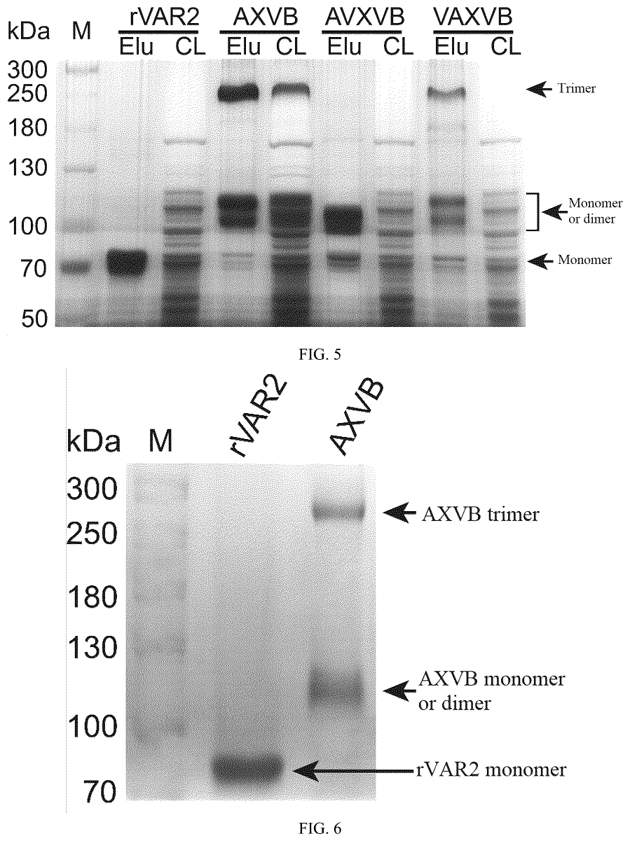

The preparation route of the catenated VAR2CSA recombinant protein is illustrated in FIG. 3, and the catenated VAR2CSA recombinant protein was synthesized intracellularly using a gene coding manner. Firstly, a protein catenation prokaryotic expression vector having a corresponding resistance selectable marker and an affinity purification tag was constructed, and the structure schematic diagram is illustrated in FIG. 4. The prokaryotic expression vector was then transferred into Escherichia coli to obtain an expression strain. After fermentation and induction of the expression strain, the catenated AXVB recombinant protein and its similar variants VAXVB and AVXVB could be formed intracellularly. Protein samples were purified with Strep-Tactin resin (IBA, Cat. #2-1201-010) and then detected by 6% sodium dodecyl sulfate polyacrylamide gel electrophoresis (SDS-PAGE), as shown in FIG. 5, where M denotes the protein molecular weight marker (Thermo Scientific, Cat. #26626), Elu denotes eluted proteins, and CL denotes cell lysates. The mean molecular weight of the rVAR2 protein (including an affinity tag and additional polypeptides introduced due to subcloning into an expression vector backbone) monomer was about 73.1 kDa; the mean molecular weight of the AXVB monomer was about 95.5 kDa; the mean molecular weight of the AVXVB monomer was about 166.2 kDa, and the protein molecular chain of the AVXVB monomer was prone to breakage; the mean molecular weight of the VAXVB monomer was about 166.5 kDa, and the protein molecular chain of the VAXVB monomer was prone to breakage; only the AXVB protein could form a trimer with stable intermolecular coupling and a high molecular weight (with a mean molecular weight of about 285.6 kDa). Meanwhile, the wild-type VAR2CSA recombinant protein (rVAR2) was purified and obtained according to the method described in CN110325551B. The rVAR2 protein and the AXVB protein which were both purified with Strep-Tactin resin were then further purified with anion-exchange resin (DEAE Beads 6FF, Cat. #S1005025) to obtain high-purity proteins and then detected by SDS-PAGE gel electrophoresis. The detection results were shown in FIG. 6, and the purity of the two proteins exceeded 95%.

The polypeptide sequence of the rVAR2 protein is SEQ ID NO. 17.

SEQ ID NO. 17:
NYIKGDPYFAEYATKLSFILNSSDANNPSEKIQKNNDEVCNCNESGIAS
VEQEQISDPSSNKTCITHSSIKANKKKVCKHVKLGVRENDKDLRVCVIE
HTSLSGVENCCCQDFLRILQENCSDNKSGSSSNGSCNNKNQEACEKNLE
KVLASLTNCYKCDKCKSEQSKKNNKNWIWKKSSGKEGGLQKEYANTIGL
PPRTQSLCLVVCLDEKGKKTQELKNIRTNSELLKEWIIAAFHEGKNLKP
SHEKKNDDNGKKLCKALEYSFADYGDLIKGTSIWDNEYTKDLELNLQKI
FGKLFRKYIKKNNTAEQDTSYSSLDELRESWWNTNKKYIWLAMKHGAGM
NSTTCCGDGSVTGSGSSCDDIPTIDLIPQYLRFLQEWVEHFCKQRQEKV
KPVIENCKSCKESGGTCNGECKTECKNKCEVYKKFIEDCKGGDGTAGSS
WVKRWDQIYKRYSKYIEDAKRNRKAGTKNCGPSSTTNAAENKCVQSDID
SFFKHLIDIGLTTPSSYLSIVLDDNICGADKAPWTTYTTYTTTEKCNKE
TDKSKLQQCNTAVVVNVPSPLGNTPHGYKYACQCKIPTNEETCDDRKEY
MNQWSCGSARTMKRGYKNDNYELCKYNGVDVKPTTVRSNSSKLD.

### Example 3

In this example, T cell sorting, transduction with a CAR-expressing lentivirus and in vitro expansion of CAR-T cells were performed.

The peripheral blood donated by healthy volunteers was obtained according to the standard venous blood collection process, and all processes must be in accordance with the Chinese Doctor of Ethics.

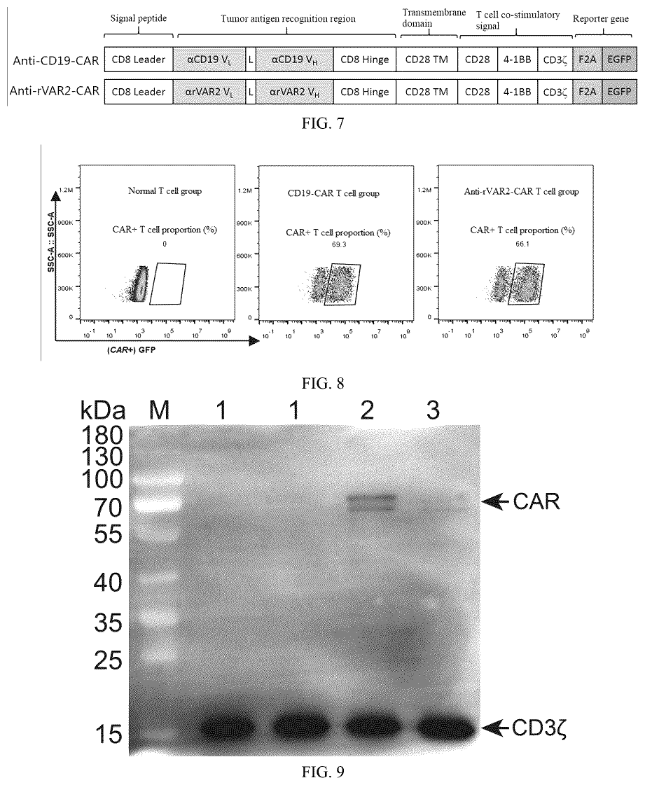

Peripheral blood mononuclear cells (PBMCs) were first isolated by density gradient centrifugation using LymphoPrep reagent (purchased from Stemcell, Cat. #07851). The PBMCs were sorted by magnetic bead sorting according to the standard experimental protocols provided in the instructions of Dynabeads® CD8 Positive Isolation Kit (purchased from Invitrogen, Cat. #11333D) and Dynabeads® CD4 Positive Isolation Kit (purchased from Invitrogen, Cat. #11331D) to sequentially obtain CD8⁺ T cells and CD4⁺ T cells. The sorted CD8⁺ T cells and CD4⁺ T cells were counted and mixed in a ratio of 1:1 to form a T cell suspension. CD3/CD28 co-stimulatory magnetic beads were added to the T cell suspension in a ratio of 1:2 according to the instructions of Dynabeads Human T-Expander CD3/CD28 (purchased from Invitrogen, Cat. #11332D), and the T cells were stimulated overnight in X-VIVO-15 (Lonza, Cat. #BE02-060F) complete medium (supplemented with 10% fetal bovine serum (Biological Industries, Cat. #04-001-1ACS), 2 mM L-glutamine (Gibco, Cat. #25030-081) and 210 IU/mL recombinant human interleukin-2 (rhIL-2, R&D System, Cat. #202-IL-050)). T cell transduction was performed on the stimulated T cells with a lentivirus packaged by a lentiviral expression vector (pLentiCART-anti-rVAR2) coding Anti-rVAR2-CAR described in CN110325551B according to the experimental steps described in CN110325551B. Before analysis, the T cells in the control groups (the normal T cell group and the Anti-CD19-CAR T cell group) and the T cells in the experimental group (the Anti-rVAR2-CAR T cell group, expressing the chimeric antigen receptor shown in SEQ ID NO. 16) were subjected to medium exchange with X-VIVO-15 complete medium three times per week and continuously cultured for 14 days. The module compositions of the chimeric antigen receptors (CARs) in the Anti-CD19-CAR T (usually referred to as CD19-CAR T) cell group and the Anti-rVAR2-CAR T cell group were illustrated in FIG. 7, where CD8 leader denotes a membrane expression signal peptide, αCD19 V_{L} denotes the light chain of an anti-CD19 single-chain fragment variable antibody, L denotes a flexible linker sequence, αCD19 V_{H} denotes the heavy chain of an anti-CD19 single-chain fragment variable antibody, CD8 Hinge denotes an extracellular CD8 hinge region, CD28 TM denotes a CD28 transmembrane domain, CD28 and 4-1BB denote T cell co-stimulatory signals, CD3ζ denotes a T cell activation signal, F2A denotes a self-cleavable “2A” peptide, EGFP denotes an enhanced green fluorescence protein as a reporter gene, αrVAR2 V_{L} denotes the light chain of an anti-VAR2CSA recombinant protein single-chain fragment variable antibody, and αrVAR2 V_{H} denotes the heavy chain of an anti-VAR2CSA recombinant protein single-chain fragment variable antibody.

The proportion (positive rate) of CAR⁺-T cells was detected by flow cytometry before CAR-T cell infusion (FIG. 8), which was about 70%. The reserved T cell samples were then detected by western-blot using the horseradish peroxidase (HRP)-labelled CD3ζ antibody (6B10.2, SANTA CRUZ, Cat. #sc-1239 HRP) to detect CAR expression, as shown in FIG. 9, where lane 1 denotes normal T cells, lane 2 denotes Anti-rVAR2-CAR T cells, and lane 3 denotes CD19-CAR T cells. The mean molecular weight of Anti-rVAR2-CAR was about 75 kDa, the mean molecular weight of CD19-CAR was about 73 kDa, and the mean molecular weight of the constitutive CD3ζ expressed in all T cells was about 16 kDa. The results show that all the molecular weights obtained by western-blot meet expectations, indicating that the CAR expression is normal.

The chimeric antigen receptor (CAR) in the Anti-CD19-CAR T (hereinafter referred to as CD19-CAR T) cells was consistent with Anti-rVAR2-CAR shown in SEQ ID NO. 16 in terms of the polypeptide sequence and functional domain arrangement, except that the sequence of the single-chain fragment variable (ScFv) antibody (Anti-CD19 ScFv) was different. Both the light and heavy chains of the Anti-CD19 ScFv were derived from the murine monoclonal antibody FMC63 (Nicholson et al. Mol Immunol. 1997, 34(16-17):1157-65) that is specific for human CD19, and the Anti-CD19 ScFv has a polypeptide sequence shown in SEQ ID NO. 18.

SEQ ID NO. 18:
QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCAR
RETTTVGRYYYAMDYWGQGTTVTVSSGGGGSGGGGSGGGGSDIQLTQSP
ASLAVSLGQRATISCKASQSVDYDGDSYLNWYQQIPGQPPKLLIYDASN
LVSGIPPRFSGSGSGTDFTLNIHPVEKVDAATYHCQQSTEDPWTFGGGT
KLEIK.

### Example 4

In this example, the affinities of the catenated VAR2CSA recombinant proteins (AXVB, VAXVB and AVXVB) and the wild-type VAR2CSA recombinant protein (rVAR2) for single-chain fragment variable antibody in the extracellular recognition domain of CAR-T cells were compared.

In the present application, the single-chain fragment variable antibody in the extracellular recognition domain of the CAR-T cells was 5H4 ScFv (PCT/CN2017/113661, ZL201780001820.4), which is composed of the V_{H} chain and the V_{L} chain of the monoclonal antibody 5H4 (5H4 mAb) that recognizes the ID2α epitope in the domains of the catenated VAR2CSA recombinant proteins (AXVB, VAXVB and AVXVB) and the wild-type VAR2CSA recombinant protein (rVAR2) as well as a connection sequence (SEQ ID NO. 19: GGGGSGGGGSGGGGS) between the V_{H} chain and the V_{L} chain. The dissociation constant K_{d} (affinity constant) of the monoclonal antibody 5H4 (5H4 mAb) for each of the catenated VAR2CSA recombinant proteins (AXVB, VAXVB and AVXVB) and the wild-type VAR2CSA recombinant protein (rVAR2) was determined by ELISA (Syedbasha et al., J Vis Exp. 2016, (109):53575) to indirectly compare the strength of the affinities of the catenated VAR2CSA recombinant proteins (AXVB, VAXVB and AVXVB) and the wild-type VAR2CSA recombinant protein (rVAR2) with 5H4 ScFv located in the extracellular recognition domain of the CAR-T cells.

The specific steps are as follows:
(1) Antigen coating: The AXVB and rVAR2 proteins were diluted to a final concentration of 20 nM with the Na₂CO₃—NaHCO₃ carbonate coating buffer (with a pH of 9.6) which was filtered and sterilized by the 0.22 μm filter membrane, respectively, 100 μL of each protein sample was added to a 96-well Elisa plate (JET BIOFIL, Guangzhou, China, Cat. #FEP101896), with three replicates per protein sample, and the Elisa plate was covered with a sealing film and incubated at 4° C. overnight.
(2) Antigen blocking: The coating solution in the Elisa plate was removed, the Elisa plate was washed with a PBST solution (1×PBS+0.05% v/v polyoxyethylene sorbitan fatty acid ester, with a pH of 7.2-7.4) three times, the liquid was patted dry, 200 μL of PBST solution containing 5% BSA was added, and the Elisa plate was then incubated at room temperature for 2 hours.
(3) Primary antibody incubation: The antigen blocking solution was removed, the Elisa plate was washed with the PBST solution three times, and 100 μL of 5H4 mAb at different concentrations which was diluted with an antibody dilution solution (1×PBS containing 0.1% BSA) in different ratios was added to each well. Only 100 μL of antibody dilution solution was added to a negative control well, and an equal volume of 1:1000 diluted murine anti-rVAR2 protein polyvalent antiserum was taken as a positive control well, with three replicates per negative and positive control. The Elisa plate was then incubated at room temperature for one hour.
(4) Secondary antibody incubation: After the primary antibody incubation was completed, the Elisa plate was washed with the PBST solution three times, 100 μL of HRP-labelled goat anti-mouse IgG (H+L) secondary antibody (Invitrogen, Cat. #31430) diluted with the antibody dilution solution in a ratio of 1:10000 was added, and the Elisa plate was incubated at room temperature for 45 minutes.
(5) TMB substrate addition for color development: After the secondary antibody incubation was completed, the Elisa plate was washed with the PBST solution three times, 100 μL of freshly prepared TMB substrate color developing agent (prepared by mixing the equilibrated TMB substrate A and B solutions at room temperature in a ratio of 1:1) was added to each well, color development was performed at room temperature in the dark for 20 minutes, and after the color development was completed, 50 μL of 2 M sulphuric acid was then added to each well to terminate the reaction.
(6) Elisa plate reading and data analysis: The absorbance (optical density, OD₄₅₀) values were directly read at 450 nm using a BioTek Synergy H1 microplate reader, and the data were analyzed using GraphPad Prism software.

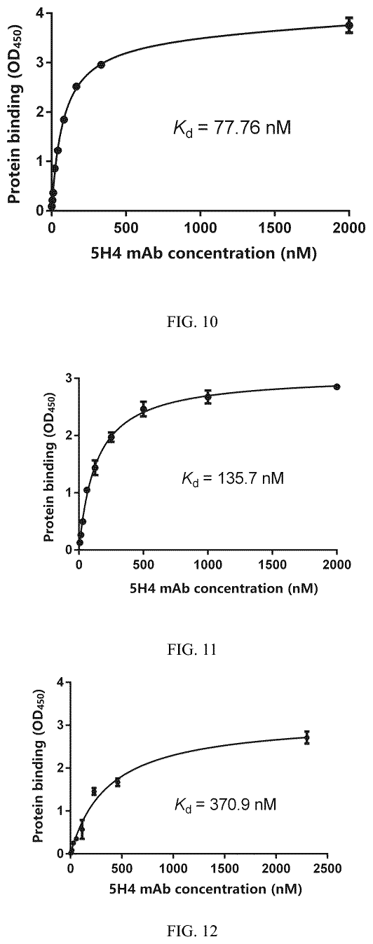

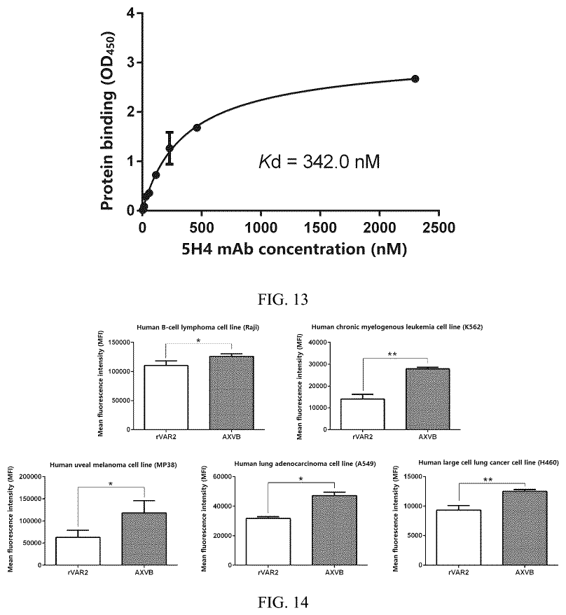

The ELISA results were analyzed by non-linear regression fitting curves with the following experimental data as an example: the equilibrium dissociation constant K_{d} of 5H4 mAb for the AXVB protein is 77.76 nM (FIG. 10) and the dissociation constant K_{d} of 5H4 mAb for the rVAR2 protein is 135.7 nM (FIG. 11). The experiments were repeated three times. The results show that the mean dissociation constant K_{d}=83.66 nM (73.03 nM, 77.76 nM, and 100.2 nM) of 5H4 mAb for the AXVB protein was less than the mean dissociation constant K_{d}=144.07 nM (122.8 nM, 135.7 nM, and 173.7 nM) of 5H4 mAb for the rVAR2 protein, which indicates that the affinity of the AXVB protein for 5H4 mAb was higher than the affinity of the rVAR2 protein for 5H4 mAb and which also indirectly proves that the AXVB protein had a stronger affinity for the single-chain fragment variable antibody (5H4 ScFv) constituting the CAR than the rVAR2 protein. The other two catenated protein variants AVXVB (FIG. 12) and VAXVB (FIG. 13) had weaker affinity for 5H4 mAb than the AXVB protein. Therefore, the AXVB protein was selected as the preferred object for subsequent function comparison studies with the rVAR2 protein.

### Example 5

In this example, the binding affinities of the AXVB protein and the rVAR2 protein for tumor cells were compared.

According to the experiment results in Example 2 and Example 4, through comparison, the catenated VAR2CSA recombinant protein AXVB had a higher yield and higher affinity for the single-chain fragment variable antibody 5H4 ScFv of the CAR-T cells, and therefore, the AXVB protein was selected for further comparative tests.

2×10⁵ in vitro cultured tumor cells of different types were separately incubated at 25° C. with 0.2 μM AXVB protein, 0.2 μM rVAR2 protein, 1 μg/mL mouse anti-rVAR2 protein monoclonal antibody 5H4 mAb and 1 μg/mL Alexa Fluor® 488-labelled goat anti-mouse IgG (H&L) secondary antibody (Abcam, Cat. #ab150113), and all of the above proteins and antibodies were diluted with the antibody diluent (1×PBS containing 0.1% FBS). After one protein or antibody was incubated for 45 minutes, the protein or antibody was washed with pre-cooled PBS-F (containing 0.02% NaN₃ and 2% FBS) at 4° C. three times before the next protein or antibody was incubated or after the secondary antibody incubation was completed, at intervals of 2 minutes each. Finally, the relative mean fluorescence intensities (MFI) of the tumor cell lines incubated with the recombinant proteins AXVB and rVAR2 were detected by BD Accuri™ C6 Plus flow cytometry to compare the strength of the cell-binding affinities of the proteins AXVB and rVAR2. As shown in the results in FIG. 14, statistically, the AXVB protein had a significantly stronger binding affinity for tumor cells than the rVAR2 protein (p<0.05 or p<0.01).

### Example 6

In this example, the thermal stabilities of the AXVB protein and the rVAR2 protein were compared.

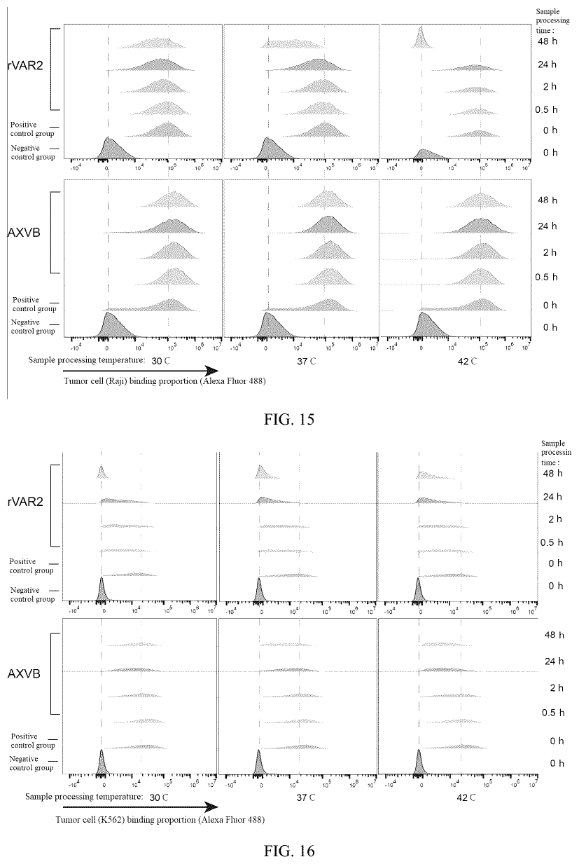

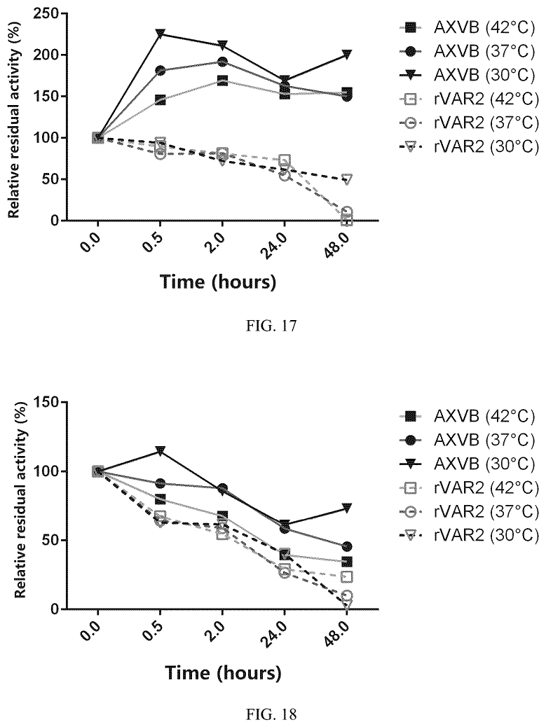

Cytokine release syndrome (CRS) is the most common side effect of CAR-T cell therapy (Neelapu S S. Hematol Oncol. 2019; 37(S1):48-52), and the CRS at the lowest grade can cause a fever ≥38° C. or even a prolonged high fever (≥39° C.) for more than 10 hours, which greatly exceeds the normal temperature (36.1° C.-37.2° C.) of the human body. Therefore, the thermal stability of the protein may directly affect the efficacy of the CAR-T cell system using the protein as the navigation system in human clinical tumor treatment. In the present application, the thermal stability of the navigator protein was assessed by dynamically monitoring the binding activity with the tumor cells through the following steps.
(1) A frozen AXVB or rVAR2 protein with a purity of ≥98% was taken from a refrigerator (at −80° C.), thawed on ice, adjusted to a protein concentration of 200 nM with an ice-cooled PBS buffer and divided into four tubes, 500 μL per tube. The samples were then separately incubated for 30 minutes, 2 hours, 24 hours, and 48 hours in the temperature conditions of 30° C., 37° C. and 42° C. The incubated samples were periodically collected, and the same batch of purified AXVB or rVAR2 protein stored at −80° C. was thawed on ice and then taken as a positive control protein for further researches.
(2) 2×10⁵ Raji cells (a human B-cell lymphoma cell line, ATCC #CCL86) or K562 cells (a human chronic myelogenous leukemia cell line, ATCC #CCL-243) were collected and incubated with 200 μL each protein sample of 200 nM AXVB or rVAR2 recombinant protein collected in different temperature conditions and at different time points from step (1), 1 μg/mL mouse anti-rVAR2 protein monoclonal antibody 5H4 mAb and 1 μg/mL Alexa Fluor® 488-labelled goat anti-mouse IgG (H&L) secondary antibody (purchased from Abcam, Cat. #ab150113), and all of the above proteins and antibodies were diluted with the antibody diluent (1×PBS containing 0.1% FBS). After one protein or antibody was incubated for 45 minutes, the protein or antibody was washed with pre-cooled PBS-F (containing 0.02% NaN₃ and 2% FBS) at 4° C. for three times before the next protein or antibody was incubated or after the secondary antibody incubation was completed, at intervals of 2 minutes each. An equal number of cells that were not incubated with recombinant proteins were taken as the negative control groups.
(3) The positive rates and corresponding mean fluorescence intensities (MFIs) of the recombinant proteins AXVB and rVAR2 after binding to Raji (FIG. 15) or K562 (FIG. 16) were detected by Cytek Aurora flow cytometry. As shown in FIG. 15, with the increase in the processing temperature and processing time, the binding proportion of the AXVB protein to Raji cells was significantly higher than the binding proportion of the rVAR2 protein to Raji cells, and the rVAR2 protein completely lost its binding activity to tumor cells after 48 hours of processing at 42° C. As shown in FIG. 16, with the increase in the processing temperature and processing time, the binding proportion of the AXVB protein to K562 cells was significantly higher than the binding proportion of the rVAR2 protein to Raji cells, and the rVAR2 protein almost completely lost its binding activity to tumor cells after 24 hours of processing at 30° C. or above. We further analyzed the difference in the time-dependent relative residual activity of the proteins after binding to tumor cells Raji (Table 1, FIG. 17) or K562 (Table 1, FIG. 18) in different temperature conditions, in which the relative residual activity (%)=(MFI of experimental group samples/MFI of positive control group samples)×100. The thermal stabilities of the AXVB and rVAR2 proteins can be indirectly compared by comparing the difference in the relative residual activity of the AXVB and rVAR2 proteins binding to tumor cells.

TABLE 1

| | | | | | Mean | Relative |
|---|---|---|---|---|---|---|
| | | | | | fluorescence | residual |
| | | | Temperature | Processing | intensity | activity |
| Group | Cell | Protein | (° C.) | time (h) | (MFI) | (%) |
| Experimental | Raji | rVAR2 | 30.0 | 48.0 | 39563 | 49.3 |
| group | Raji | rVAR2 | 30.0 | 24.0 | 49548 | 61.7 |
| | Raji | rVAR2 | 30.0 | 2.0 | 57939 | 72.1 |
| | Raji | rVAR2 | 30.0 | 0.5 | 75550 | 94.1 |
| Positive | Raji | rVAR2 | 0.0 | 0.0 | 80319 | 100.0 |
| control | | | | | | |
| group | | | | | | |
| Negative | Raji | / | 0.0 | 0.0 | 2209 | 2.8 |
| control | | | | | | |
| group | | | | | | |
| Experimental | Raji | rVAR2 | 37.0 | 48.0 | 8756 | 10.9 |
| group | Raji | rVAR2 | 37.0 | 24.0 | 44112 | 54.9 |
| | Raji | rVAR2 | 37.0 | 2.0 | 65511 | 81.6 |
| | Raji | rVAR2 | 37.0 | 0.5 | 64922 | 80.8 |
| Positive | Raji | rVAR2 | 0.0 | 0.0 | 80319 | 100.0 |
| control | | | | | | |
| group | | | | | | |
| Negative | Raji | / | 0.0 | 0.0 | 2209 | 2.8 |
| control | | | | | | |
| group | | | | | | |
| Experimental | Raji | rVAR2 | 42.0 | 48.0 | 723 | 0.9 |
| group | Raji | rVAR2 | 42.0 | 24.0 | 58809 | 73.2 |
| | Raji | rVAR2 | 42.0 | 2.0 | 65314 | 81.3 |
| | Raji | rVAR2 | 42.0 | 0.5 | 71956 | 89.6 |
| Positive | Raji | rVAR2 | 0.0 | 0.0 | 80319 | 100.0 |
| control | | | | | | |
| group | | | | | | |
| Negative | Raji | / | 0.0 | 0.0 | 2209 | 2.8 |
| control | | | | | | |
| group | | | | | | |
| Experimental | Raji | AXVB | 30.0 | 48.0 | 138398 | 200.1 |
| group | Raji | AXVB | 30.0 | 24.0 | 117027 | 169.2 |
| | Raji | AXVB | 30.0 | 2.0 | 146094 | 211.2 |
| | Raji | AXVB | 30.0 | 0.5 | 155728 | 225.1 |
| Positive | Raji | AXVB | 0.0 | 0.0 | 69173 | 100.0 |
| control | | | | | | |
| group | | | | | | |
| Negative | Raji | / | 0.0 | 0.0 | 2209 | 3.2 |
| control | | | | | | |
| group | | | | | | |
| Experimental | Raji | AXVB | 37.0 | 48.0 | 103871 | 150.2 |
| group | Raji | AXVB | 37.0 | 24.0 | 112691 | 162.9 |
| | Raji | AXVB | 37.0 | 2.0 | 132800 | 192.0 |
| | Raji | AXVB | 37.0 | 0.5 | 125441 | 181.3 |
| Positive | Raji | AXVB | 0.0 | 0.0 | 69173 | 100.0 |
| control | | | | | | |
| group | | | | | | |
| Negative | Raji | / | 0.0 | 0.0 | 2209 | 3.2 |
| control | | | | | | |
| group | | | | | | |
| Experimental | Raji | AXVB | 42.0 | 48.0 | 107173 | 154.9 |
| group | Raji | AXVB | 42.0 | 24.0 | 105839 | 153.0 |
| | Raji | AXVB | 42.0 | 2.0 | 117027 | 169.2 |
| | Raji | AXVB | 42.0 | 0.5 | 100992 | 146.0 |
| Positive | Raji | AXVB | 0.0 | 0.0 | 69173 | 100.0 |
| control | | | | | | |
| group | | | | | | |
| Negative | Raji | / | 0.0 | 0.0 | 2209 | 3.2 |
| control | | | | | | |
| group | | | | | | |
| Experimental | K562 | rVAR2 | 30.0 | 48.0 | 296 | 2.8 |
| group | K562 | rVAR2 | 30.0 | 24.0 | 4265 | 40.2 |
| | K562 | rVAR2 | 30.0 | 2.0 | 6533 | 61.6 |
| | K562 | rVAR2 | 30.0 | 0.5 | 6666 | 62.8 |
| Positive | K562 | rVAR2 | 0.0 | 0.0 | 10607 | 100.0 |
| control | | | | | | |
| group | | | | | | |
| Negative | K562 | / | 0.0 | 0.0 | 366 | 3.5 |
| control | | | | | | |
| group | | | | | | |
| Experimental | K562 | rVAR2 | 37.0 | 48.0 | 1057 | 10.0 |
| group | K562 | rVAR2 | 37.0 | 24.0 | 2813 | 26.5 |
| | K562 | rVAR2 | 37.0 | 2.0 | 6245 | 58.9 |
| | K562 | rVAR2 | 37.0 | 0.5 | 6817 | 64.3 |
| Positive | K562 | rVAR2 | 0.0 | 0.0 | 10607 | 100.0 |
| control | | | | | | |
| group | | | | | | |
| Negative | K562 | / | 0.0 | 0.0 | 366 | 3.5 |
| control | | | | | | |
| group | | | | | | |
| Experimental | K562 | rVAR2 | 42.0 | 48.0 | 2518 | 23.7 |
| group | K562 | rVAR2 | 42.0 | 24.0 | 3096 | 29.2 |
| | K562 | rVAR2 | 42.0 | 2.0 | 5779 | 54.5 |
| | K562 | rVAR2 | 42.0 | 0.5 | 7175 | 67.6 |
| Positive | K562 | rVAR2 | 0.0 | 0.0 | 10607 | 100.0 |
| control | | | | | | |
| group | | | | | | |
| Negative | K562 | / | 0.0 | 0.0 | 366 | 3.5 |
| control | | | | | | |
| group | | | | | | |
| Experimental | K562 | AXVB | 30.0 | 48.0 | 13916 | 73.3 |
| group | K562 | AXVB | 30.0 | 24.0 | 11640 | 61.3 |
| | K562 | AXVB | 30.0 | 2.0 | 16297 | 85.8 |
| | K562 | AXVB | 30.0 | 0.5 | 21730 | 114.4 |
| Positive | K562 | AXVB | 0.0 | 0.0 | 18993 | 100.0 |
| control | | | | | | |
| group | | | | | | |
| Negative | K562 | / | 0.0 | 0.0 | 366 | 1.9 |
| control | | | | | | |
| group | | | | | | |
| Experimental | K562 | AXVB | 37.0 | 48.0 | 8659 | 45.6 |
| group | K562 | AXVB | 37.0 | 24.0 | 11122 | 58.6 |
| | K562 | AXVB | 37.0 | 2.0 | 16700 | 87.9 |
| | K562 | AXVB | 37.0 | 0.5 | 17368 | 91.4 |
| Positive | K562 | AXVB | 0.0 | 0.0 | 18993 | 100.0 |
| control | | | | | | |
| group | | | | | | |
| Negative | K562 | / | 0.0 | 0.0 | 366 | 1.9 |
| control | | | | | | |
| group | | | | | | |
| Experimental | K562 | AXVB | 42.0 | 48.0 | 6548 | 34.5 |
| group | K562 | AXVB | 42.0 | 24.0 | 7500 | 39.5 |
| | K562 | AXVB | 42.0 | 2.0 | 12882 | 67.8 |
| | K562 | AXVB | 42.0 | 0.5 | 15195 | 80.0 |
| Positive | K562 | AXVB | 0.0 | 0.0 | 18993 | 100.0 |
| control | | | | | | |
| group | | | | | | |
| Negative | K562 | / | 0.0 | 0.0 | 366 | 1.9 |
| control | | | | | | |
| group | | | | | | |

The above results suggest that the AXVB protein has better thermal stability than the rVAR2 protein, which may help to enhance the long-lasting activity of the AXVB protein in the human body, thereby improving the anti-tumor efficacy of the CAR-T cell system using the AXVB protein as the navigation system.

### Example 7

In this example, the in vitro cytotoxicity of different CAR-T cell systems against tumor cells was compared.

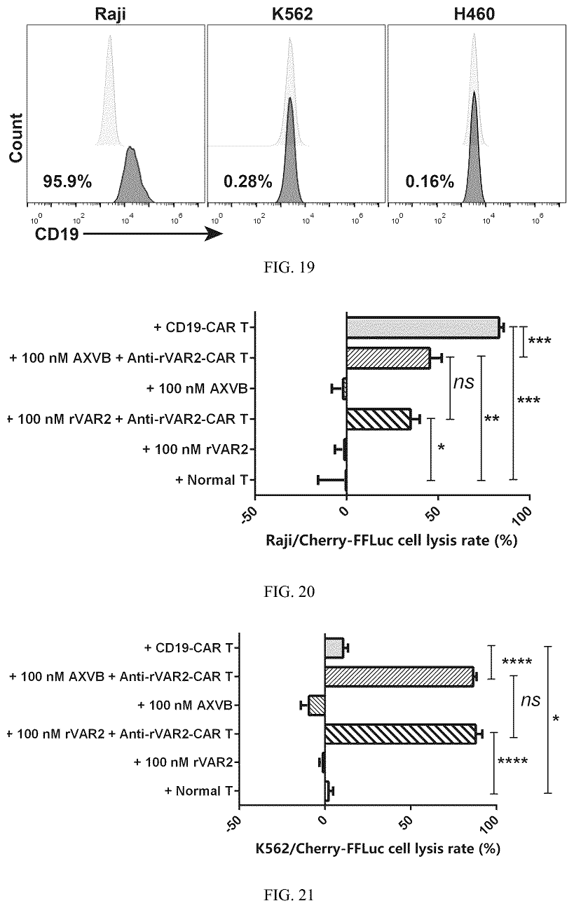

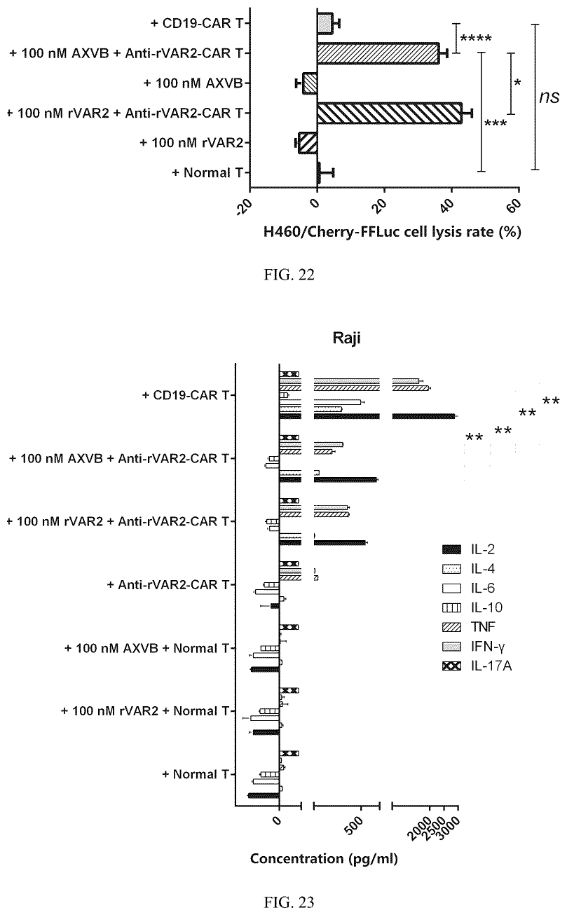

The cytotoxicity of the CAR-T cell system (AXVB-[switch]-CAR T) using the AXVB protein as the navigation system was validated by standard luciferase activity assay (Eyquem et al., Nature. 2017, 543(7643):113-117). Briefly, the stable transfected cell lines Raji/mCherry-FFLuc (CD19-positive hematological tumor cells), K562/mCherry-FFLuc (CD19-negative hematological tumor cells) and H460/mCherry-FFLuc (a human large cell lung cancer cell line, ATCC #HTB177, CD19-negative solid tumor cells) stably expressing the fusion protein of red fluorescence protein (mCherry) and firefly luciferase (FFLuc) were selected as target cells, and the flow cytometry analysis results of the target cells are shown in FIG. 19. With a total volume of the cell culture solution maintained at 100 μL, 1×10⁵ target cells were co-cultured with the corresponding effector cell system (CART-anti-rVAR2 cells and 100 nM AXVB protein) according to an E/T ratio of effector cells (effector, E) to target cells (Tumor target, T)=4:1 in a 96-well black-walled cell culture plate using a 1640 complete medium (a phenol red-free RPMI-1640 medium (Gibco, Cat. #11875093) containing 10% inactivated FBS (Biological Industries, Cat. #04-001-1ACS)), with three replicates per sample. The target cells alone were plated at the same cell density to determine the maximum luciferase expression intensity (relative luminescence unit; RLUₘₐₓ). 24 hours later, 100 μL of luciferase substrate (Bright-Glo, Promega, Cat. #E2650) was added directly to each cell culture well. The emitted light was detected in a BioTek Synergy H1 system, and the data were analyzed using GraphPad Prism software. The cell lysis rate (%) was calculated by the formula (1−(RLUₛₐₘₚₗₑ)/(RLUₘₐₓ))×100. The in vitro cytotoxicity assay results of different target cell lines are shown in FIG. 20 (Raji/mCherry-FFLuc), FIG. 21 (K562/mCherry-FFLuc) and FIG. 22 (H460/mCherry-FFLuc). As shown in the graph, AXVB-[switch]-CAR T was cytotoxic in vitro to many different types of tumor cells. For CD19-positive Raji/mCherry-FFLuc cells, compared to the normal T cell group (Normal T), CD19-CAR T had the highest cytotoxicity, AXVB-[switch]-CAR T and rVAR2-[switch]-CAR T also had significant cytotoxicity (FIG. 20), while the AXVB-[switch]-CAR T system had higher cytotoxicity; both AXVB-[switch]-CAR T and rVAR2-[switch]-CAR T had significantly higher in vitro cytotoxicity compared to the normal T-cell group (Normal T) (FIG. 20, where p<0.05 and p<0.05, respectively). Interestingly, CD19-CAR T also showed some in vitro cytotoxicity to CD19-negative K562/mCherry-FFLuc cells (p<0.05, FIG. 21), which may be caused by a non-specific killing effect due to activation during CAR-T preparation. For the non-small cell lung cancer cell line H460/mCherry-FFLuc, both AXVB-[switch]-CAR T and rVAR2-[switch]-CAR T had significant cytotoxicity compared to the normal T-cell group (Normal T) (p<0.001, FIG. 22), and the cytotoxicity of the AXVB-[switch]-CAR T system was slightly weaker than the cytotoxicity of the rVAR2-[switch]-CAR T system, which indicates that the AXVB-[switch]-CAR T system may have different therapeutic effects on hematological tumors and solid tumors. The above data (FIGS. 20 to 22) suggest that (1) compared to the CD19-CAR T, AXVB-[switch]-CAR T and rVAR2-[switch]-CAR T have a broader anti-tumor effect; (2) neither the rVAR2 protein nor the AXVB protein has in vitro cytotoxicity when used alone; (3) under the conditions of this assay, the in vitro cytotoxicity of the AXVB-[switch]-CAR T system and the rVAR2-[switch]-CAR T system to tumor cells is comparable, probably due to the excess of both AXVB and rVAR2 proteins in the system, which does not fully reflect the advantages of the AXVB protein that the AXVB protein has more binding sites and higher affinity with tumor cells.

### Example 8

In this example, the in vitro cytokine secretion level of CAR-T cells was measured by CBA assay.

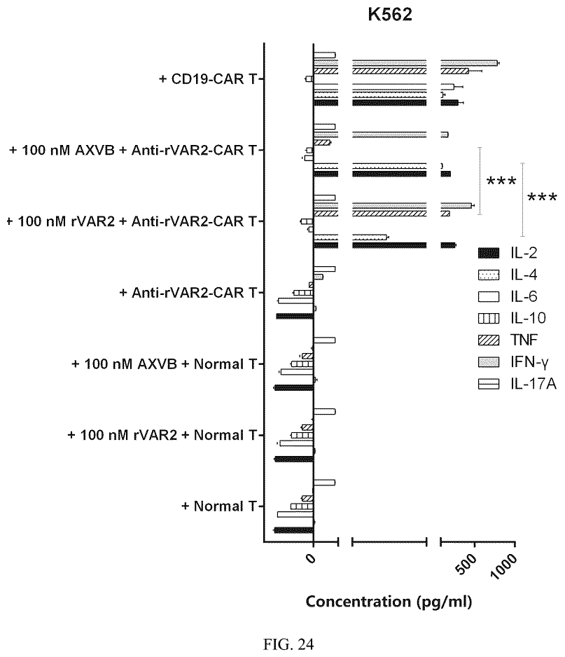

The BD™ Cytometric Bead Array (CBA) Human Th1/Th2/Th17 Cytokine Kit (BD, Cat. #560484) was used, and the kit can simultaneously detect the secretion levels of a total of seven cytokines of AXVB-[switch]-CAR T or sCART-anti-rVAR2 (that is, rVAR2-[switch]-CAR T) in a single sample, including Interleukin-2 (IL-2), Interleukin-4 (IL-4), Interleukin-6 (IL-6), Interleukin-10 (IL-10), Tumor Necrosis Factor (TNF), Interferon-γ (IFN-γ) and Interleukin-17A (IL-17A). The main assay steps are as follows:
(1) In a 96-well cell culture plate, the cultured Raji (ATCC #CCL86) cells and the cultured K562 (ATCC #CCL-243) cells, as target cells, were resuspended with a certain amount of 1640 complete medium, mixed gently and spread at a density and volume of “5000 cells/100 μL of medium/well”, the rVAR2 protein or the AXVB protein with a final concentration of 100 nM was separately added to the normal T cell group (Normal T) and the Anti-rVAR2-CAR T group, and the plate was then incubated at 37° C. in a 5% CO₂ incubator.
(2) 3 hours later, an appropriate number of “CAR⁺-T cells/100 μL of medium/well” were added according to an effector-to-target ratio (E/T=4:1) and incubated at 37° C. in a 5% CO₂ incubator for 24 hours. The same number of normal T cells resuspended with 100 μL of RPMI-1640 complete medium were added to the normal T-cell control group and incubated at the same conditions.
(3) Standard samples and cell culture supernatant samples were prepared separately for flow cytometry according to the instructions of the CBA cytokine kit (BD, Cat. #560484). The samples were detected by Cytek Aurora flow cytometer, and the data were parsed by SpectroFlo software of the flow cytometer and then analyzed by Microsoft Excel and GraphPad Prism software. The conclusions were as follows: (1) compared to CD19-CAR T, AXVB-[switch]-CAR T and rVAR2-[switch]-CAR T secreted lower levels of cytokines (FIG. 23 and FIG. 24), in particular, the secretion of pro-inflammatory cytokines such as IL-6, TNF and IFN-γ was at a significantly low level (p<0.01), and the anti-inflammatory cytokine IL-10 was hardly secreted and the anti-inflammatory cytokine IL-4 was also at a low level in all the other experimental groups except for the CD19-CAR T cell group, which indicates the lower risk of cytokine release syndrome (CRS); (2) the secretion of effector cytokines such as IL-2, IFN-γ and IL-6 relied on the presence of a certain concentration of navigator proteins in the system (FIG. 23 and FIG. 24), which indicates that the cytokine secretion level of CAR-T cells as well as their cytotoxicity to tumor cells can be effectively regulated by regulating the concentration of the navigator protein, so that the AXVB-[switch]-CAR T cell system is functionally controllable as the rVAR2-[switch]-CAR T cell system; (3) T cells in all control and experimental groups secreted the Th17 cytokine IL-17A, and there was no significant difference in the secretion level of IL-17A (FIG. 23 and FIG. 24, p>0.05), which indicates that the prepared CAR-T cells are capable of surviving for a longer period of time in the tumor microenvironment; (4) for specific types of tumor cells (for example, K562), compared to the rVAR2-[switch]-CAR T cell system (FIG. 24), the AXVB-[switch]-CAR T cell system secreted a significantly lower level of the systemic pro-inflammatory cytokine TNF (p<0.001) and also secreted a significantly higher level of the anti-inflammatory cytokine IL-4 (p<0.001), which indicates that the AXVB-[switch]-CAR T cell system is less prone to cytokine release syndrome in vivo and has better safety.

### Example 9

In this example, the functional controllability of the CAR-T cell system using the catenated VAR2CSA recombinant protein as the navigation system is tested.

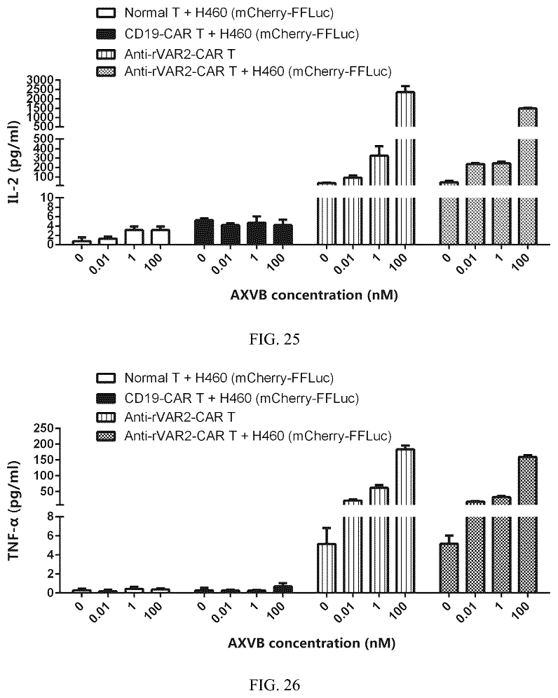

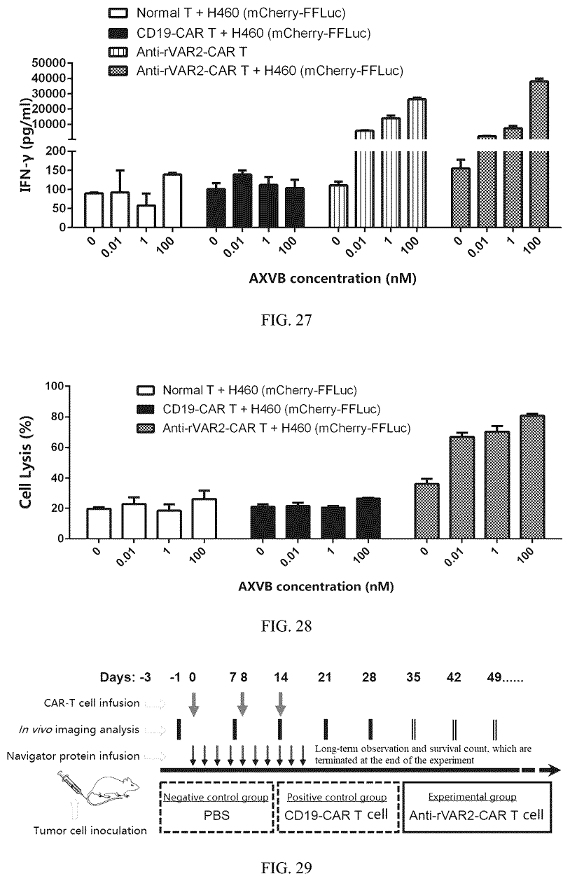

To verify whether the catenated VAR2CSA recombinant protein can regulate the function of CAR-T cells when used as the navigation system of the CAR-T cell system, the regulation role of the navigator protein “switch” in the CAR-T cell system was analyzed in terms of cytokine secretion and cytotoxicity of the CAR-T cell system. Firstly, the regulation of the AXVB navigator protein on the cytokine secretion level in the AXVB-[switch]-CAR T cell system was detected by ELISA, and the specific steps are as follows:
(1) Sample preparation: The cultured cells were resuspended with RPMI-1640 (Gibco, Cat. #11875093) complete medium (containing 3% inactivated fetal bovine serum) containing a navigator protein at different molar concentration gradients, and tumor cells H460 (mCherry-FFLuc) were subsequently inoculated at a density of 1.25×10⁵ cells/250 μL/well in a 48-well cell culture plate (Costar, Product #3548), with three replicates per navigator protein concentration. After 3 hours of incubation at 37° C. in a 5% CO₂ incubator for 3 hours, the plate was centrifuged at 300×g for 5 minutes at room temperature to remove the supernatant, and 250 μL of new RPMI-1640 complete medium was added with a pipette. 250 μL of normal T cells and 250 μL of CAR-T cells were separately added according to an E/T ratio of T cells (effector cells, E) to tumor cells (target cells, T)=1:1 and co-cultured, the total numbers of the two T cells were identical, and the sum of target cells and effector cells was not more than 1×10⁶ cells/500 μL/well. The background level of cytokine secretion for the experimental groups was represented by the Anti-rVAR2-CAR T cell group, and in this group, we only added 250 μL of Anti-rVAR2-CAR T cells and 250 μL of RPMI-1640 complete medium containing a navigator protein at different molar concentration gradients. Finally, all the experimental samples were co-cultured at 37° C. in a 5% CO₂ incubator.
(2) Sample collection: 24 hours later, the test samples were transferred to a clean and sterile 1.5 mL EP tube and centrifuged at 500×g for 5 minutes at room temperature, and 300 μL of cell culture supernatant was aspirated with a pipette and transferred to a new clean and sterile 1.5 mL EP tube for subsequent ELISA assays.
(3) The contents of cytokines IL-2 (R&D Systems, Cat. #D2050), TNF-α (R&D Systems, Cat. #DTA00D) and IFN-γ (R&D Systems, Cat. #DIF50) in the collected cell culture supernatant were detected according to the instructions of an R&D ELISA Kit, respectively, and the data were analyzed using GraphPad Prism software. The detection results of IL-2 are shown in FIG. 25, and compared to the secretion levels of IL-2 in the normal T-cell control group and the CD19-CAR T-cell group, the secretion level of IL-2 in the AXVB-[switch]-CAR T-cell system was directly proportional to the concentration of the catenated protein AXVB in the system. The detection results of TNF-α and IFN-γ are shown in FIG. 26 and FIG. 27, respectively, and similarly, compared to the secretion levels of IL-2 in the normal T-cell control group and the CD19-CAR T-cell group, the secretion level of IL-2 in the AXVB-[switch]-CAR T-cell system was also proportional to the concentration of the catenated protein AXVB in the system. The above results suggest that the cytokine secretion level in the AXVB-[switch]-CAR T-cell system can be regulated by regulating the content of the catenated VAR2CSA recombinant protein AXVB.

Secondly, using the same method as in Example 7, the cytotoxicity of navigator protein-mediated CAR-T cells against the human non-small cell lung cancer cell line H460/mCherry-FFLuc at different molar concentration gradients were compared to indirectly analyze the role of the AXVB navigator protein in regulating the anti-tumor activity of the AXVB-[switch]-CAR T cell system. The experiment results are shown in FIG. 28. Compared to the cytotoxicity against tumor cells in the normal T-cell control group and the CD19-CAR T-cell group, the cytotoxicity of the AXVB-[switch]-CAR T-cell system against tumor cells was proportional to the concentration of the catenated protein AXVB in the system, which indicates that the killing activity of the AXVB-[switch]-CAR T cell system against tumor cells can be regulated by regulating the content of the catenated VAR2CSA recombinant protein AXVB.

As shown in the FIG. 25 to 28, the function of the CAR T cell system (for example, AXVB-[switch]-CAR T) using the catenated VAR2CSA recombinant protein as the navigation system can be regulated by increasing or decreasing the content of the navigator protein in the system. The catenated VAR2CSA recombinant protein not only has a navigation function but also acts as a “safety switch”.

### Example 10

In this example, the in vivo anti-tumor activity of the CAR-T cell system was detected through animal model experiments.

To validate and compare the in vivo anti-tumor activity of the AXVB-[switch]-CAR T cell system, in vivo experiments in an animal model were designed and implemented, and the implementation process is shown in FIG. 29. Firstly, female NSG mice (NOD.Cg-Prkdcscid Il2rgtm1Wjl/SzJ, purchased from Beijing Biocytogen Co., Ltd), 8-10 weeks old, were inoculated with 5×10⁵ stable transduced cells of Raji/mCherry-FFLuc and K562/mCherry-FFLuc via tail-vein injections respectively, to construct tumor-bearing mouse models. On the third day (Day −3) post-inoculation, the luciferase expression levels of the Raji/mCherry-FFLuc or K562/mCherry-FFLuc tumor-bearing mice were measured by in vivo imaging. The mice with close expression levels were mixed and randomly grouped into different groups, and a total number of up to 2×10⁷ CAR-T cells (60%-80% CAR⁺ positive rate) were infused into the mice via tail vein injections in a single infusion on Days 0, 8 and 14. 3 hours later, the 100 nmol/kg of rVAR2 or AXVB navigator protein “switch” was infused into each mouse in the Anti-rVAR2-CAR T cell treatment group, followed by the infusion of the navigator protein every other day for a total of 10 infusions. From the day prior (Day −1) to the CAR-T cell infusion, the mice were imaged in vivo using the IVIS-Spectrum imaging system (Caliper Life Sciences, Hopkinton, MA, USA) and detected a total of five times at an average interval of approximately 7 days. All the mice to be examined by in vivo imaging were anesthetized with isoflurane (2%) and injected intraperitoneally (i.p.) with D-Luciferin (purchased from Yeasen Biotechnology (Shanghai) Co., Ltd) at a concentration of 150 mg/kg (D-Luciferin quality/body weight), imaging was performed 12 minutes after D-Luciferin injection. The luciferase activity was measured in photons per second per square centimeter per steradian (p/s⁻¹cm²sr⁻¹). Image analysis was performed using Living Image software (Caliper Life Sciences, Hopkinton, MA, USA). The body weights of the mice were monitored from the day prior to the CAR-T infusion (Day −1) and measured twice a week. If the body weight of a tumor-bearing mouse was ≥20% lighter than its body weight before injection during the experiment or the body weight of a tumor-bearing mouse decreased drastically by ≥15% in a short period of time, which means that an adverse reaction occurred, the tumor-bearing mouse would be euthanized, and the mice with ulcerated tumors would also be euthanized. All animal experiments were conducted in strict compliance with the “3R” principle of animal welfare and were approved by the Laboratory Animal Ethics Review Committee of the research unit.

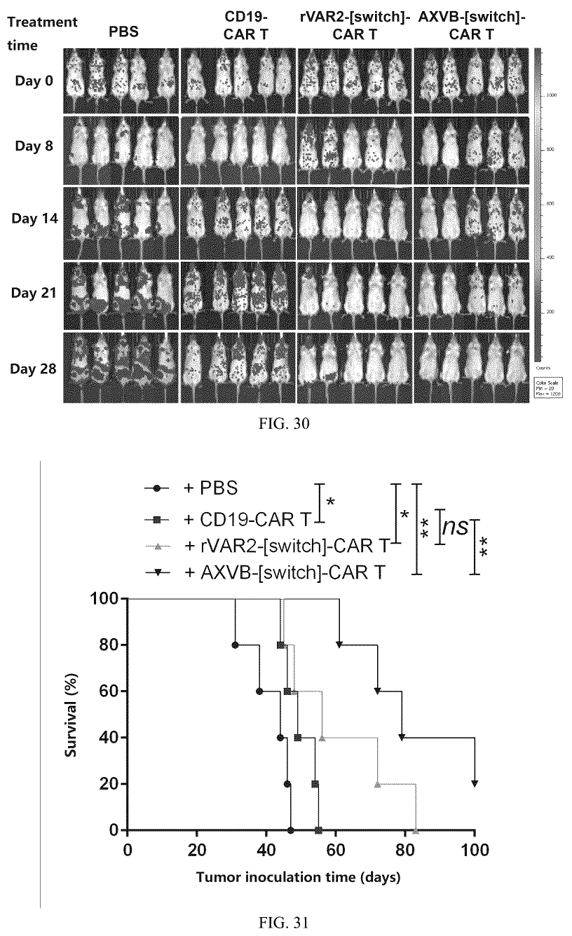

The results of in vivo imaging experiments in the tumor-bearing mice showed that for CD19-positive Raji cells, compared with the untreated control group (infused with only an equal volume of 1×PBS used for resuspending T cells), both AXVB-[switch]-CAR T and rVAR2-[switch]-CAR T, as well as CD19-CAR T, all of which could consistently reduce the tumor load (FIG. 30) from Day 8 after CAR-T cell infusion, and the survival of the mice was significantly prolonged (FIG. 31), with the longest median survival of 79 days in AXVB-[switch]-CAR T treated group, compared to 44 days for PBS treated group, 49 days for CD19-CAR T treated group and 56 days for rVAR2-[switch]-CAR T treated group.

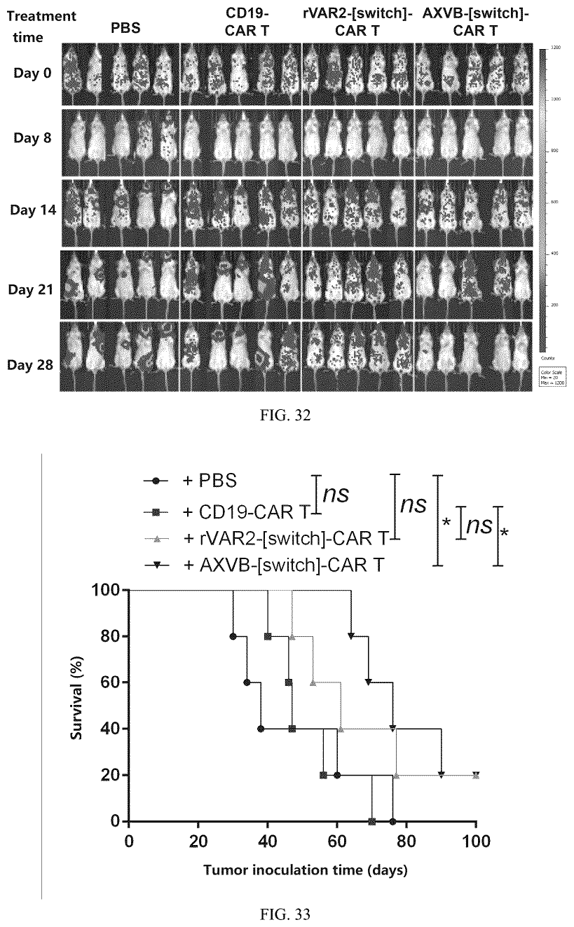

For CD19-negative K562 cells, AXVB-[switch]-CAR T, rVAR2-[switch]-CAR T were as effective as CD19-CAR T in reducing tumor load from Day 8 after CAR-T cell infusion compared with untreated control group (PBS, with only 1×PBS buffer for resuspension of T cells). However, with time goes on, by Day 14 post-treatment, tumor growth in mice in the CD19-CAR T treated group began to rebound, and the tumor load in the late stage was almost identical to that of the untreated control group (FIG. 32). AXVB-[switch]-CAR T treated mice had the longest median survival time and was significantly different from the survival of the PBS control group (p<0.05, FIG. 33). The median survival of the mice in the AXVB-[switch]-CAR T group reached 76 days, while the median survival of the tumor-bearing mice in other groups was 38 days (PBS), 47 days (CD19-CAR T) and 61 days (rVAR2-[switch]-CAR T), respectively. The above results suggest that AXVB-[switch]-CAR T and rVAR2-[switch]-CAR T have a broader anti-tumor spectrum than CD19-CAR T, and the AXVB-[switch]-CAR T has a better in vivo anti-tumor effect.

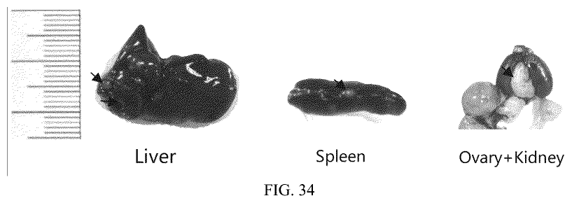

Furthermore, the anatomical observations of some Raji cell-bearing mice after euthanasia revealed that tumor metastases were detected in the liver, spleen and ovary tissues of some mice in the PBS control group (FIG. 34), whereas no metastasis was detected in other CAR-T cell treated groups, suggesting that the metastasis of the tumor cells to the vital organs via the blood circulation and the lymph circulation could be effectively inhibited in the CAR-T cell treated groups.

In summary, in the present application, a catenated VAR2CSA recombinant protein is designed and successfully prepared, and compared to a common VAR2CSA recombinant protein (wild-type), the catenated VAR2CSA recombinant protein is significantly improved in terms of the protein stability and the protein affinity for the tumor-specific antigen placenta-like chondroitin sulfate A. A chimeric antigen receptor cell system is creatively assembled and designed, and the catenated VAR2CSA recombinant protein is applied to immune cell therapy. In the chimeric antigen receptor cell system, the chimeric antigen receptor cell uses the catenated VAR2CSA recombinant protein as the navigation system (for example, AXVB-[switch]-CAR T), and compared to a chimeric antigen receptor cell system using the wild-type VAR2CSA recombinant protein as the navigation system (for example, rVAR2-[switch]-CAR T), the catenated VAR2CSA navigating chimeric antigen receptor cell system herein has a stronger and more durable anti-tumor activity and a better in vivo anti-tumor effect. Furthermore, the chimeric antigen receptor cell system using the catenated VAR2CSA recombinant protein as the navigation system can obtain or lose the anti-tumor activity by supplying or cutting off the catenated VAR2CSA recombinant protein in the system, and the function of the chimeric antigen receptor cell system can also be regulated by regulating the content of the catenated VAR2CSA recombinant protein in the system. The catenated VAR2CSA recombinant protein in the system acts like an adjustable “safety switch” to enable the whole chimeric antigen receptor cell system to be controllable and enhance the safety of the system.

The applicant has stated that although the detailed method of the present application is described through the examples described above, the present application is not limited to the detailed method described above, which means that the implementation of the present application does not necessarily depend on the detailed method described above. It is to be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

The instant application contains a Sequence Listing which has been submitted electronically in ASCII text file format and is hereby incorporated by reference in its entirety. Said ASCII text file created on Sep. 5, 2024, is named SequenceListing.txt and is 69,292 bytes in size.

1. A catenated VAR2CSA recombinant protein, comprising binding domain, SpyTag, a p53dim domain and SpyCatcher;
wherein the binding domain comprises a domain, in a VAR2CSA protein, binding to placental-like chondroitin sulfate A;
a polypeptide sequence of the catenated VAR2CSA recombinant protein is a sequence shown in SEQ ID NO. 8.

2. A method for preparing the catenated VAR2CSA recombinant protein of claim 1, comprising:
constructing an expression vector comprising a coding gene of the catenated VAR2CSA recombinant protein of claim 1, transfecting the expression vector into cells, culturing the cells, and performing protein purification to obtain the catenated VAR2CSA recombinant protein.

3. A nucleic acid molecule, which is a coding gene of the catenated VAR2CSA recombinant protein of claim 1;
wherein the coding gene of the catenated VAR2CSA recombinant protein is a deoxyribonucleic acid sequence shown in SEQ ID NO. 11 or a variant thereof having at least 80% nucleotide identity or more.

4. A chimeric antigen receptor cell system, comprising the catenated VAR2CSA recombinant protein of claim 1 and a chimeric antigen receptor cell;
wherein the chimeric antigen receptor cell expresses a chimeric antigen receptor that recognizes the catenated VAR2CSA recombinant protein;
the cell is an immune effector cell, and the immune effector cell comprises any one or a combination of at least two of T cells, B cells, NK cells, NKT cells, dendritic cells or macrophages.

5. The chimeric antigen receptor cell system of claim 4, wherein the chimeric antigen receptor comprises a domain recognizing the catenated VAR2CSA recombinant protein;
the chimeric antigen receptor further comprises a hinge region, a transmembrane domain and an intracellular co-stimulatory signaling domain;
the domain recognizing the catenated VAR2CSA recombinant protein comprises a single-chain fragment variable antibody composed of a heavy chain variable region and a light chain variable region;
a gene coding the heavy chain variable region of the single-chain fragment variable antibody is a deoxyribonucleic acid sequence shown in SEQ ID NO. 14;
a gene coding the light chain variable region of the single-chain fragment variable antibody is a deoxyribonucleic acid sequence shown in SEQ ID NO. 15;
the hinge region is a human CD8α hinge region;
the transmembrane domain is a human CD28 transmembrane domain;
the intracellular co-stimulatory signaling domain is any one or a combination of at least two of a human CD27 intracellular signaling domain, a human CD134 intracellular signaling domain, a human CD28 intracellular signaling domain or a human 4-1BB intracellular signaling domain;
an amino-terminus of the chimeric antigen receptor comprises one CD8α signal peptide;
a carboxyl-terminus of the chimeric antigen receptor comprises a human CD3ζ intracellular signaling domain;
the chimeric antigen receptor comprises a CD8α signal peptide, a single-chain fragment variable antibody recognizing the catenated VAR2CSA recombinant protein, a human CD8α hinge region, a human CD28 transmembrane domain, a human CD28 intracellular signaling domain, a human 4-1BB intracellular signaling domain and a human CD3ζ intracellular signaling domain that are in tandem sequentially from an N-terminus to a C-terminus of a protein;
the chimeric antigen receptor is a polypeptide sequence shown in SEQ ID NO. 16.

6. A chimeric antigen receptor cell, expressing a chimeric antigen receptor that recognizes the catenated VAR2CSA recombinant protein of claim 1.

7. A pharmaceutical composition, comprising any one or a combination of at least two of the catenated VAR2CSA recombinant protein of claim 1, a nucleic acid molecule, a chimeric antigen receptor cell system or a chimeric antigen receptor cell;
wherein the pharmaceutical composition further comprises the pharmaceutically acceptable adjuvants;
wherein the nucleic acid molecule is a coding gene of the catenated VAR2CSA recombinant protein of claim 1; and the coding gene of the catenated VAR2CSA recombinant protein is a deoxyribonucleic acid sequence shown in SEQ ID NO. 11 or a variant thereof having at least 80% nucleotide identity or more;
wherein the chimeric antigen receptor cell system comprises the catenated VAR2CSA recombinant protein of claim 1 and a chimeric antigen receptor cell; and the chimeric antigen receptor cell expresses and recognizes a chimeric antigen receptor of the catenated VAR2CSA recombinant protein; and the cell is an immune effector cell, and the immune effector cell comprises any one or a combination of at least two of a T cell, a B cell, an NK cell, a dendritic cell or a macrophage;
wherein the chimeric antigen receptor cell expresses and recognizes a chimeric antigen receptor of the catenated VAR2CSA recombinant protein of claim 1.

8. (canceled)

9. A method for treating a tumor, comprising administering an effective amount of the catenated VAR2CSA recombinant protein of claim 1 to subject in need thereof, wherein the tumor is a solid tumor and/or a hematological tumor expressing placental-like chondroitin sulfate A.
